SEQUENCE LISTING (10 sequences)

SEQ ID NO: 1 - protein, 119 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 2 - protein, 107 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 3 - protein, 449 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 213 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGA

SEQ ID NO: 5 - protein, 8 aa, synthetic construct
GFSLTNYG

SEQ ID NO: 6 - protein, 5 aa, synthetic construct
WSGGN

SEQ ID NO: 7 - protein, 7 aa, synthetic construct
LTYYDYE

SEQ ID NO: 8 - protein, 7 aa, synthetic construct
SQSIGTN

SEQ ID NO: 9 - protein, 5 aa, synthetic construct
KYASE

SEQ ID NO: 10 - protein, 6 aa, synthetic construct
NNNWPT

(19) United States
(12) Patent Application Publication
(10) Pub. No.: US 2026/0207603 A1
(43) Pub. Date: Jul. 23, 2026

(54) COMBINATION THERAPY FOR CANCER TREATMENT

(71) Applicant: Hoffmann-La Roche Inc., Little Falls, NJ (US)

(72) Inventors: Jan ECKMANN, Muenchen (DE); Piergiorgio Francesco Tommaso PETTAZZONI, Basel (CH); Gabriel SCHNETZLER, Basel (CH); Juergen WICHMANN, Steinen (DE); Pascal Jean Claude DOTT, Rixheim (FR); Serena Maria FANTASIA, Saint-Louis (FR); Maud GUILLEMOT-PLASS, Village-Neuf (FR); Dainis KALDRE, Lörrach (DE); Giacomo Marco LARI, Basel (CH); Isabelle Georgette Huguette MODOLO-CHELLAT, Riehen (CH); Joerg Mathias SEDELMEIER, Basel (CH); Sebastian TROKOWSKI, Giebenach (CH)

(73) Assignee: Hoffmann-La Roche Inc., Little Falls, NJ (US)

(21) Appl. No.: 19/138,422

(22) PCT Filed: Dec. 14, 2023
(86) PCT No.: PCT/EP2023/085770

§ 371 Date: Jun. 12, 2025

(30) Foreign Application Priority Data
Dec. 15, 2022 (EP) 22213814.1
Oct. 13, 2023 (EP) 23203565.9

(51) Int. Cl. A61K 31/517; A61K 31/4745; A61K 31/513; A61K 31/519; A61K 31/555; A61K 39/395; A61K 45/06; A61P 35/00

(52) U.S. Cl. CPC A61K 31/517; A61K 31/4745; A61K 31/513; A61K 31/519; A61K 31/555; A61K 39/3955; A61K 45/06; A61P 35/00

(57) ABSTRACT

Provided herein are combination therapies comprising a BRAF inhibitor (e.g., a compound of formula (I), or a pharmaceutically acceptable salt thereof) and an EGRF inhibitor (EGRFi), as well as pharmaceutical compositions, methods and uses thereof.

### TECHNICAL FIELD

The present invention relates to a combination of a compound of formula (I):

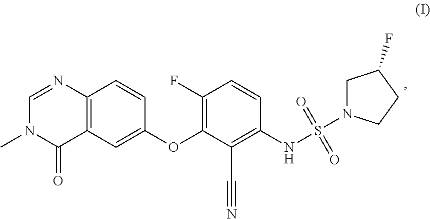 [SMILES: Cn1cnc2ccc(Oc3c(F)ccc(NS(=O)(=O)N4CC[C@@H](F)C4)c3C#N)cc2c1=O]

or a pharmaceutically acceptable salt thereof, and an EGFR inhibitor (EGFRi), as well as pharmaceutical compositions, methods and uses thereof, wherein the compound of formula (I) is a BRAF inhibitor.

### SEQUENCE LISTING

This application incorporates by reference the Computer Readable Form (CRF) of a Sequence Listing in ASCII text format submitted via EFS-Web. The Sequence Listing text file submitted via EFS-Web, entitled P38651_SEQ LISTING_ST26.xml, with a file size of 10,068 bytes, created on Oct. 3, 2023.

### BACKGROUND

Mutant BRAF is a targetable oncogenic driver and three BRAF inhibitors (BRAFi) up to date (Vemurafenib, Dabrafenib Encorafenib) reached the market showing efficacy in BRAFV600E-positive melanoma. However, rapid acquisition of drug resistance is almost universally observed and the duration of the therapeutic benefits for the targeted therapy remains limited.

Moreover, the developed first generation BRAF inhibitors revealed an unexpected and “paradoxical” ability to repress MAPK signalling in BRAF^{V600}E-driven tumors while the same inhibitors presented MAPK stimulatory activities in BRAF wild type (WT) models (N. Engl. J. Med., 366:271-273 (2012); and British Journal of Cancer, volume 111, pages 640-645(2014)).

Mechanistic studies on the RAF paradox then clarified that oncogenic BRAF^{V600E} phosphorylates MEK 1/2 in its monomeric cytosolic form while WT BRAF and RAF1 activation requires a complex step of events including cell membrane translocation and homo and/or heterodimerization promoted by activated RAS (KRAS, NRAS, HRAS) (Nature Reviews Cancer, volume 14, pages 455-467 (2014)).

The binding of first generation BRAF inhibitors like Vemurafenib, Dabrafenib and Encorafenib to a WT BRAF or RAF1 protomer, quickly induces RAF homo and/or hetero dimerization and membrane association of the newly formed RAF dimer. In the dimeric conformation, one RAF protomer allosterically induces conformational changes of the second resulting in a kinase active status and, importantly, in a conformation unfavourable for the binding of the inhibitor. The dimer induced by drug treatment, as a result, promotes MEK phosphorylation by the catalysis operated by the unbound protomer with hyperactivation of the pathway.

In Colorectal Cancer (CRC) several preclinical and clinical data demonstrated that signaling from EGFR contribute in maintenance of the MAPK activity and contribute to partial activation of RAF dimer formation through the EGFR-RAS pathway. EGFR blockade through anti-EGFR antibodies have provided some clinical benefit in combination with first generation BRAF inhibition (N. Engl. J. Med., 381:1632-1643 (2019), DOI: 10.1056/NEJMoa1908075). Albeit this combination proved some clinical success, the benefit from the paradox inducing BRAFi and anti EGFR antibodies remain limited. Due to dose-limiting toxicities of the first generation BRAF inhibitors, C_{trough} levels that cover IC90 or above are thus difficult to achieve in this clinical setting.

### SUMMARY OF THE DISCLOSURE

Provided herein are solutions to these and other problems in the art.

In one aspect, the present invention relates to novel combination therapy and uses of a compound of formula (I):

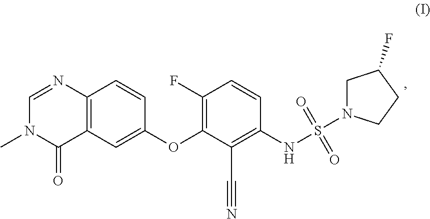 [SMILES: Cn1cnc2ccc(Oc3c(F)ccc(NS(=O)(=O)N4CC[C@@H](F)C4)c3C#N)cc2c1=O]

or a pharmaceutically acceptable salt thereof. In some embodiments, the compound of formula (I) is (3R)-N-[2-cyano-4-fluoro-3-(3-methyl-4-oxo-quinazolin-6-yl)oxy-phenyl]-3-fluoro-pyrrolidine-1-sulfonamide, or a pharmaceutically acceptable salt thereof. In some embodiments, the compound of formula (I), or the pharmaceutically acceptable salt thereof, is a BRAy inhibitor. The compound of formula (I), for example, is also described in the International Patent Publication No. WO2021116050A1 and is also referred to as R07276389.

In another aspect, the present invention relates to a new combination of a compound of formula (I) and an EGFR inhibitor, as well as said combination for use in the treatment of cancer, in particular of colorectal cancer. The compound of formula (I) is a BRAF inhibitor, which is, for example, showing neglectable paradoxical activation of the MAPK signaling pathway (paradox breaker) when compared to the first generation BRAF inhibitors that are on the market: Encorafenib, Dabrafenib and Vemurafenib (paradox inducers). In another example, the compound of formula (I) also has very potent brain penetration properties, thus providing an urgently needed alternative therapy for the treatment of leptomeningial cancer or cancers which metastasized in the brain. By virtue of an excellent safety profile, the compound of formula (I) allows for a much higher individual dose reaching significantly higher Ct_{rough} levels around IC95.

In another aspect, the present invention discloses a new combination for use in cancer therapy with strong combined activity on BRAF associated tumours with, for example, the potential to overcome the rapidly acquired treatment resistance frequently observed in patients treated with first generation BRAF inhibitors. In one example, the combination as disclosed in the present invention for use in the treatment of cancer, presents unexpected combination activity that go beyond the additive effects of BRAF inhibitor and EGFRi monotherapies.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 illustrates a schematic representation of the study design reported in FIG. 2 to and specifies the dosage regime that was used for treating the different cohorts in a colorectal tumour model bearing LS411N xenografts, according to some embodiments. The same study design was, for example, also used for studying a colorectal model bearing HT29 xenografts as reported in FIGS. 6 to 9. Both models are CRC models and present the BRAF V600E mutation.

FIG. 2 reports in vivo anti-tumor activity of a monotherapy of either R07276389 at 20 mpk (miligrams of compound per kilogram of body weight), 50 mpk or 140 mpk, or of encorafenib at 24 mpk on LS411N xenograft tumors in BALB/c Nude mice (10 mice/cohort), according to some embodiments. Mice were, for example, treated (QD) until day 43 and the graphs express mean tumour volume [mm3] for the different cohorts.

FIG. 3 reports in vivo anti-tumor activity of a combination of R07276389 at 140 mpk or of encorafenib at 24 mpk, in each instance with 20 mpk of cetuximab on LS411N xenograft tumors in BALB/c Nude mice (10 mice/cohort), according to some embodiments. Monotherapy controls were, for example, also included and the mice were treated (QD) until day 43 and the graph expresses mean tumour volume [mm3] for the different cohorts. The results are displayed in linear scale (top; FIG. 3a) and in logarithmic scale (bottom; FIG. 3b).

FIG. 4 reports in vivo anti-tumor activity of a combination of R07276389 at 60 mpk or of encorafenib at 24 mpk, in each instance with 20 mpk of cetuximab on LS411N xenograft tumors in BALB/c Nude mice (10 mice/cohort), according to some embodiments. Monotherapy controls were, for example, also included and the mice were treated (QD) until day 43 and the graph expresses mean tumour volume [mm3] for the different cohorts. The results are displayed in linear scale (top; FIG. 4a) and in logarithmic scale (bottom; FIG. 4b).

FIG. 5 reports in vivo anti-tumor activity of a combination of R07276389 at 30 mpk or of encorafenib at 24 mpk, in each instance with 20 mpk of cetuximab on LS411N xenograft tumors in BALB/c Nude mice (10 mice/cohort), according to some embodiments. Monotherapy controls were, for example, also included and the mice were treated (QD) until day 43 and the graph expresses mean tumour volume [mm3] for the different cohorts. The results are displayed in linear scale (top; FIG. 5a) and in logarithmic scale (bottom; FIG. 5b).

FIG. 6 reports in vivo anti-tumor activity of a monotherapy of either R07276389 at 20 mpk, 50 mpk or 140 mpk, or of encorafenib at 24 mpk on HT29 xenograft tumors in BALB/c Nude mice (10 mice/cohort), according to some embodiments. Mice were, for example, treated (QD) until day 43 and the graph expresses mean tumour volume [mm3] for the different cohorts. The results are displayed in linear scale (top; FIG. 6a) and in logarithmic scale (bottom; FIG. 6b).

FIG. 7 reports in vivo anti-tumor activity of a combination of R07276389 at 140 mpk or of encorafenib at 24 mpk, in each instance with 20 mpk of cetuximab on HT29 xenograft tumors in BALB/c Nude mice (10 mice/cohort), according to some embodiments. Monotherapy controls were, for example, also included and the mice were treated (QD) until day 43 and the graph expresses mean tumour volume [mm3] for the different cohorts. The results are displayed in linear scale (top; FIG. 7a) and in logarithmic scale (bottom; FIG. 7b).

FIG. 8 reports in vivo anti-tumor activity of a combination of R07276389 at 60 mpk or of encorafenib at 24 mpk, in each instance with 20 mpk of cetuximab on HT29 xenograft tumors in BALB/c Nude mice (10 mice/cohort), according to some embodiments. Monotherapy controls were, for example, also included and the mice were treated (QD) until day 43 and the graph expresses mean tumour volume [mm3] for the different cohorts. The results are displayed in linear scale (top; FIG. 8a) and in logarithmic scale (bottom; FIG. 8b).

FIG. 9 reports in vivo anti-tumor activity of a combination of R07276389 at 30 mpk or of encorafenib at 24 mpk, in each instance with 20 mpk of cetuximab on HT29 xenograft tumors in BALB/c Nude mice (10 mice/cohort), according to some embodiments. Monotherapy controls were, for example, also included and the mice were treated (QD) until day 43 and the graph expresses mean tumour volume [mm3] for the different cohorts. The results are displayed in linear scale (top; FIG. 9a) and in logarithmic scale (bottom; FIG. 9b).

### DETAILED DESCRIPTION OF THE DISCLOSURE

Unless defined otherwise, all technical and scientific terms used herein have the same meaning as commonly understood by those of ordinary skill in the art to which the invention belongs. See, e.g., Singleton et al., Dictionary of Microbiology and Molecular Biology, 2nd ed., J. Wiley & Sons (New York, NY 1994); Sambrook et al., Molecular Cloning, A Laboratory Manual, Cold Springs Harbor Press (Cold Springs Harbor, NY 1989). Any methods, devices and materials similar or equivalent to those described herein can be used in the practice of this invention.

The following definitions are provided to facilitate understanding of certain terms used frequently herein and are not meant to limit the scope of the present disclosure. All references referred to herein are incorporated by reference in their entirety.

### Definitions

As used herein, and unless otherwise specified, the terms “about” and “approximately,” when referring to doses, amounts, or weight percent of ingredients of a composition or a dosage form, mean a dose, amount, or weight percent that is recognized by one of ordinary skill in the art to provide a pharmacological effect equivalent to that obtained from the specified dose, amount, or weight percent. The equivalent dose, amount, or weight percent can be within 30%, 20%, 15%, 10%, 5%, 1%, or less of the specified dose, amount, or weight percent.

The term “IC50” refers to the concentration of a particular compound required to inhibit 50% of a specific measured activity. The term “IC90” refers to the concentration of a particular compound required to inhibit 90% of a specific measured activity. The term “IC95” refers to the concentration of a particular compound required to inhibit 95% of a specific measured activity.

The term “inhibitor” denotes a compound which competes with, reduces or prevents the binding of a particular ligand to particular receptor, or which reduces or prevents the function of a particular protein. In particular, an inhibitor as used therein refers to compounds which target, decrease or inhibit activity of the respective target, particular inhibitors have an IC50 value below 1 μM, below 500 nM, below 200 nM, below 100 nM, below 50 nM, below 25 nM, below 10 nM, below 5 nM, 2 nM or below 1 nM.

The term “pharmaceutically acceptable salt” refers to those salts of the compound of formula (I) which retain the biological effectiveness and properties of the free bases or free acids, which are not biologically or otherwise undesirable. These salts can for instance be formed with inorganic acids such as hydrochloric acid, hydrobromic acid, sulfuric acid, nitric acid, phosphoric acid and the like, in particular hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein, gentisic acid and the like. In addition, these salts may be prepared by addition of an inorganic base or an organic base to the free acid. Salts derived from an inorganic base include, but are not limited to, the sodium, potassium, lithium, ammonium, calcium, magnesium salts and the like. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyimine resins and the like. Particular pharmaceutically acceptable salts of a compound of formula (I) are the hydrochloride salts, methanesulfonic acid salts and citric acid salts.

The term “FOLFOX” relates to a combination chemotherapy regimen that is used to treat colorectal cancer. It includes the drugs leucovorin calcium (folinic acid), fluorouracil, and oxaliplatin and is typically administered via intraveneous infusion. In some embodiments, the FOLFOX regimen comprises i) administering oxaliplatin in an amount of from about 50 mg/m² to about 200 mg/m²; ii) administering leucovorin in the amount of from about 200 mg/m² to about 600 mg/m²; iii) administering 5-fluororacil (5-FU) in the amount of from about 1200 mg/m² to about 3600 mg/m². FOLFOX administration is according to the prescribed and approved methods of treatment. Typically the administration of the FOLFOX regimen is spread over days 1 and 2 of a two week cycle. Modified and adjusted FOLFOX regimen are to be considered encompassed for the purpose of the present invention. FOLFOX administration in combination with administration of cetuximab can be either concurrently or subsequently, in particular subsequently.

The term “FOLFIRI” relates to a combination chemotherapy regimen that is used to treat colorectal cancer. It includes the drugs leucovorin calcium (folinic acid), fluorouracil, and irinotecan. In some embodiments, the FOLFIRI regimen comprises i) administering irinotecan in an amount of from about 150 mg/m² to about 250 mg/m², in particular about 180 mg/m²; ii) administering leucovorin in the amount of from about 200 mg/m² to about 600 mg/m²; iii) administering 5-fluororacil (5-FU) in the amount of from about 1200 mg/m² to about 3600 mg/m². FOLFIRI administration is according to the prescribed and approved methods of treatment. Typically, the administration of the FOLFIRI regimen is spread over days 1 and 2 of a two week cycle. Modified and adjusted FOLFIRI regimen ae to be considered encompassed for the purpose of the present invention. FOFIRI administration in combination with administration of cetuximab is either concurrently or subsequently, in particular subsequently.

The term “patient” refers to a human patient. A patient may be an adult.

The term “antibody” herein specifically covers monoclonal antibodies (including full-length monoclonal antibodies), polyclonal antibodies, multispecific antibodies (e.g., bispecific antibodies), and antibody fragments so long as they exhibit the desired biological activity. In one instance, the antibody is a full-length monoclonal antibody.

The term IgG “isotype” or “subclass” as used herein is meant any of the subclasses of immunoglobulins defined by the chemical and antigenic characteristics of their constant regions.

Depending on the amino acid sequences of the constant domains of their heavy chains, antibodies (immunoglobulins) can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgAQ1, and IgA2. The heavy chain constant domains that correspond to the different classes of immunoglobulins are called α, γ, ε, γ, and μ, respectively. The subunit structures and three-dimensional configurations of different classes of immunoglobulins are well known and described generally in, for example, Abbas et al., Cellular and Mol. Immunology, 4th ed. (W.B. Saunders, Co., 2000). An antibody may be part of a larger fusion molecule, formed by covalent or non-covalent association of the antibody with one or more other proteins or peptides.

The terms “full-length antibody,” “intact antibody,” and “whole antibody” are used herein interchangeably to refer to an antibody in its substantially intact form, not antibody fragments as defined below. The terms refer to an antibody comprising an Fc region.

The term “Fc region” herein is used to define a C-terminal region of an immunoglobulin heavy chain that contains at least a portion of the constant region. The term includes native sequence Fc regions and variant Fc regions. In one aspect, a human IgG heavy chain Fc region extends from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore, an antibody produced by a host cell by expression of a specific nucleic acid molecule encoding a full-length heavy chain may include the full-length heavy chain, or it may include a cleaved variant of the full-length heavy chain. This may be the case wherein the final two C-terminal amino acids of the heavy chain are glycine (G446) and lysine (K447). Therefore, the C-terminal lysine (K447), or the C-terminal glycine (G446) and lysine (K447), of the Fc region may or may not be present. Amino acid sequences of heavy chains including an Fc region are denoted herein without the C-terminal lysine (K447) if not indicated otherwise. In one aspect, a heavy chain including an Fc region as specified herein, comprised in an antibody disclosed herein, comprises an additional C-terminal glycine-lysine dipeptide (G446 and K447). In one aspect, a heavy chain including an Fc region as specified herein, comprised in an antibody disclosed herein, comprises an additional C-terminal glycine residue (G446). In one aspect, a heavy chain including an Fc region as specified herein, comprised in an antibody disclosed herein, comprises an additional C-terminal lysine residue (K447). In one embodiment, the Fc region contains a single amino acid substitution N297A of the heavy chain. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

A “naked antibody” refers to an antibody that is not conjugated to a heterologous moiety (e.g., a cytotoxic moiety) or radiolabel. The naked antibody may be present in a pharmaceutical composition.

“Antibody fragments” comprise a portion of an intact antibody, preferably comprising the antigen-binding region thereof. In some instances, the antibody fragment described herein is an antigen-binding fragment. Examples of antibody fragments include Fab, Fab′, F(ab′)₂, and Fv fragments; diabodies; linear antibodies; single-chain antibody molecules (e.g., scFvs); and multispecific antibodies formed from antibody fragments.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies in accordance with the present invention may be made by a variety of techniques, including but not limited to the hybridoma method, recombinant DNA methods, phage-display methods, and methods utilizing transgenic animals containing all or part of the human immunoglobulin loci.

The term “hypervariable region” or “HVR” as used herein refers to each of the regions of an antibody variable domain which are hypervariable in sequence and which determine antigen binding specificity, for example “complementarity determining regions” (“CDRs”).

Generally, antibodies comprise six CDRs: three in the VH (CDR-H1, CDR-H2, CDR-H3), and three in the VL (CDR-L1, CDR-L2, CDR-L3). Exemplary CDRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991)); and (c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)).

In some embodiments, CDR-H1 is referred to as HVR-H1, CDR-H2 is referred to as HVR-H2, CDR-H3 is referred to as HVR-H3, CDR-L1 is referred to as HVR-L1, CDR-L2 is referred to as HVR-L2, and CDR-L3 is referred to as HVR-L3, respectively.

Unless otherwise indicated, the CDRs are determined according to Kabat et al., supra. One of skill in the art will understand that the CDR designations can also be determined according to Chothia, supra, McCallum, supra, or any other scientifically accepted nomenclature system.

“Framework” or “FR” refers to variable domain residues other than complementary determining regions (CDRs). The FR of a variable domain generally consists of four FR domains: FR1, FR2, FR3, and FR4. Accordingly, the CDR and FR sequences generally appear in the following sequence in VH (or VL): FR1-CDR-H1(CDR-L1)-FR2-CDR-H2(CDR-L2)-FR3-CDR-H3(CDR-L3)-FR4.

The term “variable domain residue numbering as in Kabat” or “amino acid position numbering as in Kabat,” and variations thereof, refers to the numbering system used for heavy chain variable domains or light chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy chain variable domain may include a single amino acid insert (residue 52a according to Kabat) after residue 52 of H2 and inserted residues (e.g., residues 82a, 82b, and 82c, etc., according to Kabat) after heavy chain FR residue 82. The Kabat numbering of residues may be determined for a given antibody by alignment at regions of homology of the sequence of the antibody with a “standard” Kabat numbered sequence.

The term “package insert” is used to refer to instructions customarily included in commercial packages of therapeutic products, that contain information about the indications, usage, dosage, administration, combination therapy, contraindications and/or warnings concerning the use of such therapeutic products.

As used herein, “in combination with” refers to administration of one treatment modality in addition to another treatment modality, for example, a treatment regimen that includes administration of an EGFR inhibitor described herein (e.g., osimertinib or cetuximab) and Compound of formula (I), or a pharmaceutically acceptable salt thereof. As such, “in combination with” refers to administration of one treatment modality before, during, or after administration of the other treatment modality to the patient.

A drug that is administered “concurrently” with one or more other drugs is administered during the same treatment cycle, on the same day of treatment, as the one or more other drugs, and, optionally, at the same time as the one or more other drugs. For instance, for cancer therapies given every three weeks, the concurrently administered drugs are each administered on day 1 of a three-week cycle.

Non-limiting examples of EGFR inhibitors include cetuximab (Erbitux®), panitumumab (Vectibix®), osimertinib (merelectinib, Tagrisso®), erlotinib (Tarceva®), gefitinib (lressa®), necitumumab (Portrazza™), neratinib (Nerlynx®), lapatinib (Tykerb®), vandetanib (Caprelsa®) and brigatinib (Alunbrig®). Additional examples of EGFR inhibitors are known in the art. In one embodiment the EGFR inhibitor is a monoclonal anti-EGFR antibody. In one embodiment the EGFR inhibitor is an orthosteric EGFR inhibitor. In one embodiment the EGFR inhibitor is an allosteric EGFR inhibitor.

In some embodiments of the present invention the EGFR inhibitor is cetuximab (Erbitux®). Cetuximab is a chimeric monoclonal IgG1 antibody produced in a mammalian cell line (Sp2/0) by recombinant DNA technology. Cetuximab may be prepared following the methods known to the person skilled in the art and described in WO2001032712. Cetuximab is commercially available and has the following CAS Registry Number: 205923-56-4. “Cetuximab” as used herein refers to a recombinant, human/mouse chimeric monoclonal antibody that binds specifically to the extracellular domain of the human epidermal growth factor receptor (EGFR). Cetuximab is composed of the Fv regions of a murine anti-EGFR antibody with human IgG1 heavy and kappa light chain constant regions and has an approximate molecular weight of 152 k Da. Cetuximab is produced in mammalian (murine myeloma) cell culture. Cetuximab is also described in WHO Drug Information (International Nonproprietary Names for Pharmaceutical Substances), Proposed INN: List 82, Vol 13, No 4, 1999, published Dec. 9, 1999 (see page 269). In one embodiment, cetuximab is marketed under the tradename ERBITUX®.

In some embodiments of the present invention the EGFR inhibitor is panitumumab (Vectibix®). Panitumumab is a fully humanized monoclonal IgG2 antibody. Panitumumab may be prepared following the methods known to the person skilled in the art and described in WO2006069202. Panitumumab is commercially available and has the following CAS Registry Number: 339177-26-3. The term “panitumumab” refers to the antibody described in the WHO Drug Information, Vol. 18; No. 2, 2004; Proposed INN: List 91; amendment: List 96.

In one embodiment, the anti-EGFR antibody comprises:

(a) a heavy chain variable region (VH) comprising
the amino acid sequence:
(SEQ ID NO: 1)
QVQLKQSGPGLVQPSQSLSITCTVSGFSLTNYGVHWVRQSPGKGLEWLG
VIWSGGNTDYNTPFTSRLSINKDNSKSQVFFKMNSLQSNDTAIYYCARA
LTYYDYEFAYWGQGTLVTVSA,
and
(b) the light chain variable region (VL)
comprising the amino acid sequence:
(SEQ ID NO: 2)
DILLTQSPVILSVSPGERVSFSCRASQSIGTNIHWYQQRINGSPRLLIK
YASESISGIPSRFSGSGSGTDFTLSINSVESEDIADYYCQQNNNWPTTF
GAGTKLELK.

In some instances, the anti-EGFR antibody comprises (a) a VH comprising an amino acid sequence comprising having at least 95% sequence identity (e.g., at least 95%, 96%, 97%, 98%, or 99% sequence identity) to, or the sequence of SEQ ID NO: 1; (b) a VL comprising an amino acid sequence comprising having at least 95% sequence identity (e.g., at least 95%, 96%, 97%, 98%, or 99% sequence identity) to, or the sequence of SEQ ID NO: 2; or (c) a VH as in (a) and a VL as in (b).

In one embodiment, the anti-EGFR antibody comprises cetuximab, which comprises:

(a) the heavy chain amino acid sequence:
(SEQ ID NO: 3)
QVQLKQSGPGLVQPSQSLSITCTVSGFSLTNYGVHWVRQSPGKGLEWLG
VIWSGGNTDYNTPFTSRLSINKDNSKSQVFFKMNSLQSNDTAIYYCARA
LTYYDYEFAYWGQGTLVTVSAASTKGPSVFPLAPSSKSTSGGTAALGCL
VKDYFPEPVTVSWNSGALTSGVHTFPAVLQSSGLYSLSSVVTVPSSSLG
TQTYICNVNHKPSNTKVDKRVEPKSCDKTHTCPPCPAPELLGGPSVFLF
PPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKG
QPREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQK
SLSLSPGK,
and
(b) the light chain amino acid sequence:
(SEQ ID NO: 4)
DILLTQSPVILSVSPGERVSFSCRASQSIGTNIHWYQQRINGSPRLLIK
YASESISGIPSRFSGSGSGTDFTLSINSVESEDIADYYCQQNNNWPTTF
GAGTKLELKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGA.

In some embodiments, the EGFR inhibitor is an EGFR-specific antagonist that is an anti-EGFR antibody. A variety of anti-EGFR antibodies are contemplated and described herein. In certain embodiments, the isolated anti-EGFR antibody can bind to, for example, human EGFR (also referred to as ErbB-1 and HER1) as shown in UniProtKB/Swiss-Prot Accession No. P00533, or a variant thereof. In some embodiments, the anti-EGFR antibody is capable of inhibiting binding between EGF and EGFR. In some embodiments, the anti-EGFR antibody is a monoclonal antibody. In some embodiments, the anti-EGFR antibody is an antibody fragment selected from the group consisting of Fab, Fab′-SH, Fv, scFv, and (Fab′)₂ fragments. In some embodiments, the anti-EGFR antibody is a humanized antibody. In some embodiments, the anti-EGFR antibody is a chimeric antibody. In some embodiments, the anti-EGFR antibody is a human antibody. Exemplary anti-EGFR antibodies include cetuximab and panitumumab. Examples of anti-EGFR antibodies useful in the methods of this invention and methods of making them are described in U.S. Pat. Nos. 5,558,864, 6,217,866 and 7,060,808, and 7,598,350, each of which is incorporated herein by reference in its entirety.

In some embodiments, the anti-EGFR antibody comprises:
(a) an HVR-H1, HVR-H2, and HVR-H3 sequence of GFSLTNYG (SEQ ID NO: 5), WSGGN (SEQ ID NO: 6) and LTYYDYE (SEQ ID NO: 7), respectively, and
(b) an HVR-L1, HVR-L2, and HVR-L3 sequence of SQSIGTN (SEQ ID NO: 8), KYASE (SEQ ID NO: 9) and NNNWPT (SEQ ID NO: 10), respectively.
   In one embodiment, the anti-EGFR antibody comprises:

(a) a heavy chain variable region (VH) comprising
the amino acid sequence:
(SEQ ID NO: 1)
QVQLKQSGPGLVQPSQSLSITCTVSGFSLTNYGVHWVRQSPGKGLEWLG
VIWSGGNTDYNTPFTSRLSINKDNSKSQVFFKMNSLQSNDTAIYYCARA
LTYYDYEFAYWGQGTLVTVSA,
and
(b) the light chain variable region (VL)
comprising the amino acid sequence:
(SEQ ID NO: 2)
DILLTQSPVILSVSPGERVSFSCRASQSIGTNIHWYQQRTNGSPRLLIK
YASESISGIPSRFSGSGSGTDFTLSINSVESEDIADYYCQQNNNWPTTF
GAGTKLELK.

In some embodiments, the anti-EGFR antibody comprises (a) a VH comprising an amino acid sequence comprising having at least 95% sequence identity (e.g., at least 95%, 96%, 97%, 98%, or 99% sequence identity) to, or the sequence of SEQ ID NO: 1; (b) a VL comprising an amino acid sequence comprising having at least 95% sequence identity (e.g., at least 95%, 96%, 97%, 98%, or 99% sequence identity) to, or the sequence of SEQ ID NO: 2; or (c) a VH as in (a) and a VL as in (b).

In one embodiment, the anti-EGFR antibody comprises:

(a) the heavy chain amino acid sequence:
(SEQ ID NO: 3)
QVQLKQSGPGLVQPSQSLSITCTVSGFSLTNYGVHWVRQSPGKGLEWLG
VIWSGGNTDYNTPFTSRLSINKDNSKSQVFFKMNSLQSNDTAIYYCARA
LTYYDYEFAYWGQGTLVTVSAASTKGPSVFPLAPSSKSTSGGTAALGCL
VKDYFPEPVTVSWNSGALTSGVHTFPAVLQSSGLYSLSSVVTVPSSSLG
TQTYICNVNHKPSNTKVDKRVEPKSCDKTHTCPPCPAPELLGGPSVFLF
PPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKG
QPREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQK
SLSLSPGK,
and
(b) the light chain amino acid sequence:
(SEQ ID NO: 4)
DILLTQSPVILSVSPGERVSFSCRASQSIGTNIHWYQQRTNGSPRLLIK
YASESISGIPSRFSGSGSGTDFTLSINSVESEDIADYYCQQNNNWPTTF
GAGTKLELKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGA.

In some embodiments, the EGFR antibody comprises a cleavable moiety or linker that, when cleaved (e.g., by a protease in the tumor microenvironment), activates an antibody antigen binding domain to allow it to bind its antigen, e.g., by removing a non-binding steric moiety.

In some embodiments, the anti-EGFR antibody comprises the six HVR sequences (e.g., the three heavy chain HVRs and the three light chain HVRs) and/or the heavy chain variable domain and light chain variable domain from an anti-EGFR antibody described in U.S. Pat. Nos. 5,558,864, 6,217,866, 7,060,808, 7,598,350.

In a still further embodiments, the anti-EGFR antibody has reduced or minimal effector function. In a still further specific aspect, the minimal effector function results from an “effector-less Fc mutation” or a glycosylation mutation. In still a further instance, the effector-less Fc mutation is an N434S or M428L/N434S substitution in the constant region. In still a further instance, the effector-less Fc mutation is an N434S substitution in the constant region. In some instances, the isolated anti-EGFR antibody is glycosylated. Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, wherein X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used. Removal of glycosylation sites from an antibody is conveniently accomplished by altering the amino acid sequence such that one of the above-described tripeptide sequences (for N-linked glycosylation sites) is removed. The alteration may be made by substitution of an asparagine, serine or threonine residue within the glycosylation site with another amino acid residue (e.g., glycine, alanine, or a conservative substitution).

As used herein, “treating” comprises effective cancer treatment with an effective amount of a therapeutic agent (e.g., Compound of formula (I), cetuximab or panitumumab) or combination of therapeutic agents (e.g., Compound of formula (I) in combination with cetuximab or panitumumab). The treatment may be first-line treatment (e.g., the patient may be previously untreated or not have received prior systemic therapy), or second line or later treatment. For example, a patient is successfully “treated” if one or more symptoms associated with a cancer described herein are mitigated or eliminated, including, but are not limited to, reducing the proliferation of (or destroying) cancerous cells, decreasing symptoms resulting from the disease, increasing the quality of life of those suffering from the disease, decreasing the dose of other medications required to treat the disease, and/or prolonging survival of patients.

The term “delaying progression” of a disease refers to deferring, hindering, slowing, retarding, stabilizing, and/or postponing development of a cancer described herein. This delay can be of varying lengths of time, depending on the history of the cancer described herein and/or patient being treated. As is evident to one skilled in the art, a sufficient or significant delay can, in effect, encompass prevention, in that the patient does not develop the cancer.

Herein, an “effective amount” refers to the amount of a therapeutic agent described herein (e.g., Compound of formula (I) and/or cetuximab) that achieves a therapeutic result. In some examples, the effective amount of a therapeutic agent or a combination of therapeutic agents is the amount of the agent or of the combination of agents that achieves a clinical endpoint as provided herein. An effective amount herein may vary according to factors such as the disease state, age, sex, and weight of the patient, and the ability of the agent to elicit a desired response in the patient. An effective amount is also one in which any toxic or detrimental effects of the treatment are outweighed by the therapeutically beneficial effects. In some embodiments, an effective amount of the drug may have the effect in reducing the number of cancer cells; reducing the tumor size; inhibiting (i.e., slow or stop) cancer cell infiltration into peripheral organs; inhibit (i.e., slow or stop) tumor metastasis; inhibiting (i.e., slow or stop) tumor growth; and/or relieving one or more of the symptoms associated with the disease. An effective amount can be administered in one or more administrations. An effective amount of drug, compound, pharmaceutical composition, or combination therapy described herein can be an amount sufficient to accomplish therapeutic treatment either directly or indirectly.

“QD” refers to administration of an agent described herein once daily.

“BID” refers to administration of an agent described herein twice daily.

“Q2W” refers to administration of an agent described herein once every two weeks.

“PO” refers to oral administration of an agent described herein.

“IV” refers to intravenous administration of any agent described herein.

In one embodiment of the combination therapy described herein, the EGFR inhibitor is administered in accordance with a package insert.

In one embodiment of the combination therapies described herein, the EGFR inhibitor is cetuximab administered as a fixed dose Q2W administration.

In another embodiment, the combination therapy described herein comprises cetuximab, where cetuximab is administered at an amount of about 200-400 mg/m².

The compound of formula (I) contains one asymmetric center and can be present in the form of optically pure enantiomers or mixtures of enantiomers such as, for example, racemates.

According to the Cahn-Ingold-Prelog Convention the asymmetric carbon atom can be of the “R” or “S” configuration.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some aspects, the number of amino acid changes are 10 or less, 9 or less, 8 or less, 7 or less, 6 or less, 5 or less, 4 or less, 3 or less, or 2 or less. In some aspects, the VL acceptor human framework is identical in sequence to the VL human immunoglobulin framework sequence or human consensus framework sequence. “Affinity” refers to the strength of the sum total of noncovalent interactions between a single binding site of a molecule (e.g., an antibody) and its binding partner (e.g., an antigen).

Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art, including those described herein. Specific illustrative and exemplary methods for measuring binding affinity are described in the following.

An “affinity matured” antibody refers to an antibody with one or more alterations in one or more complementary determining regions (CDRs), compared to a parent antibody which does not possess such alterations, such alterations resulting in an improvement in the affinity of the antibody for antigen.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g., scFv, and scFab); single domain antibodies (dAbs); and multispecific antibodies formed from antibody fragments. For a review of certain antibody fragments, see Holliger and Hudson, Nature Biotechnology 23:1126-1136 (2005).

The term “linker” as used herein refers to a peptide linker and is preferably a peptide with an amino acid sequence with a length of at least 5 amino acids, preferably with a length of 5 to 100, more preferably of 10 to 50 amino acids. In one embodiment said peptide linker is (GₓS)ₙ or (GₓS)ₙGₘ with G=glycine, S=serine, and (x=3, n=3, 4, 5 or 6, and m=0, 1, 2 or 3) or (x=4, n=2, 3, 4 or 5 and m=0, 1, 2 or 3), preferably x=4 and n=2 or 3, more preferably with x=4, n=2. In one embodiment said peptide linker is (G₄S)₂.

The term “immunoglobulin molecule” refers to a protein having the structure of a naturally occurring antibody. For example, immunoglobulins of the IgG class are heterotetrameric glycoproteins of about 150,000 daltons, composed of two light chains and two heavy chains that are disulfide-bonded. From N- to C-terminus, each heavy chain has a variable region (VH), also called a variable heavy domain or a heavy chain variable domain, followed by three constant domains (CH1, CH2, and CH3), also called a heavy chain constant region. Similarly, from N- to C-terminus, each light chain has a variable region (VL), also called a variable light domain or a light chain variable domain, followed by a constant light (CL) domain, also called a light chain constant region. The heavy chain of an immunoglobulin may be assigned to one of five types, called α (IgA), δ (IgD), ε (IgE), γ (IgG), or μ (IgM), some of which may be further divided into subtypes, e.g. γ₁ (IgG₁), γ₂ (IgG₂), γ₃ (IgG₃), γ₄ (IgG₄), α₁ (IgA₁) and α₂ (IgA₂). The light chain of an immunoglobulin may be assigned to one of two types, called kappa (x) and lambda (k), based on the amino acid sequence of its constant domain. An immunoglobulin essentially consists of two Fab molecules and an Fc domain, linked via the immunoglobulin hinge region.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. An exemplary competition assay is provided herein.

The term “antigen binding domain” refers to the part of an antigen binding molecule that comprises the area which specifically binds to and is complementary to part or all of an antigen. Where an antigen is large, an antigen binding molecule may only bind to a particular part of the antigen, which part is termed an epitope. An antigen binding domain may be provided by, for example, one or more antibody variable domains (also called antibody variable regions). Preferably, an antigen binding domain comprises an antibody light chain variable region (VL) and an antibody heavy chain variable region (VH).

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. In certain aspects, the antibody is of the IgG₁ isotype. In certain aspects, the antibody is of the IgG₁ isotype with the P329G, L234A and L235A mutation to reduce Fc-region effector function. In other aspects, the antibody is of the IgG₂ isotype. In certain aspects, the antibody is of the IgG₄ isotype with the S228P mutation in the hinge region to improve stability of IgG₄ antibody. The heavy chain constant domains that correspond to the different classes of immunoglobulins are called α, δ, ε, γ, and μ, respectively. The light chain of an antibody may be assigned to one of two types, called kappa (x) and lambda (k), based on the amino acid sequence of its constant domain.

The terms “constant region derived from human origin” or “human constant region” as used in the current application denotes a constant heavy chain region of a human antibody of the subclass IgG₁, IgG₂, IgG₃, or IgG₄ and/or a constant light chain kappa or lambda region. Such constant regions are well known in the state of the art and e.g. described by Kabat, E. A., et al., Sequences of Proteins of Immunological Interest, 5th ed., Public Health Service, National Institutes of Health, Bethesda, MD (1991) (see also e.g. Johnson, G., and Wu, T. T., Nucleic Acids Res. 28 (2000) 214-218; Kabat, E. A., et al., Proc. Natl. Acad. Sci. USA 72 (1975) 2785-2788). Unless otherwise specified herein, numbering of amino acid residues in the constant region is according to the EU numbering system, also called the EU index of Kabat, as described in Kabat, E. A. et al., Sequences of Proteins of Immunological Interest, 5th ed., Public Health Service, National Institutes of Health, Bethesda, MD (1991), NIH Publication 91-3242.

The term “cytotoxic agent” as used herein refers to a substance that inhibits or prevents a cellular function and/or causes cell death or destruction. Cytotoxic agents include, but are not limited to, radioactive isotopes (e.g., At²¹¹, I³¹¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents or drugs (e.g., oxalilplatin, fluorouracil, methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof, and the various antitumor or anticancer agents disclosed below.

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g., B cell receptor); and B cell activation.

As used herein, the terms “engineer, engineered, engineering”, are considered to include any manipulation of the peptide backbone or the post-translational modifications of a naturally occurring or recombinant polypeptide or fragment thereof. Engineering includes modifications of the amino acid sequence, of the glycosylation pattern, or of the side chain group of individual amino acids, as well as combinations of these approaches.

The term “amino acid mutation” as used herein is meant to encompass amino acid substitutions, deletions, insertions, and modifications. Any combination of substitution, deletion, insertion, and modification can be made to arrive at the final construct, provided that the final construct possesses the desired characteristics, e.g., reduced binding to an Fc receptor, or increased association with another peptide. Amino acid sequence deletions and insertions include amino- and/or carboxy-terminal deletions and insertions of amino acids. Particular amino acid mutations are amino acid substitutions. For the purpose of altering e.g. the binding characteristics of an Fc region, non-conservative amino acid substitutions, i.e. replacing one amino acid with another amino acid having different structural and/or chemical properties, are particularly preferred. Amino acid substitutions include replacement by non-naturally occurring amino acids or by naturally occurring amino acid derivatives of the twenty standard amino acids (e.g. 4-hydroxyproline, 3-methylhistidine, ornithine, homoserine, 5-hydroxylysine). Amino acid mutations can be generated using genetic or chemical methods well known in the art. Genetic methods may include site-directed mutagenesis, PCR, gene synthesis and the like. It is contemplated that methods of altering the side chain group of an amino acid by methods other than genetic engineering, such as chemical modification, may also be useful. Various designations may be used herein to indicate the same amino acid mutation. For example, a substitution from proline at position 329 of the Fc domain to glycine can be indicated as 329G, G329, G₃₂₉, P329G, or Pro329Gly.

An “effective amount” of an agent, e.g., a pharmaceutical composition, refers to an amount effective, at dosages and for periods of time necessary, to achieve the desired therapeutic or prophylactic result.

The term “Fc region” herein is used to define a C-terminal region of an immunoglobulin heavy chain that contains at least a portion of the constant region. The term includes native sequence Fc regions and variant Fc regions. In one aspect, a human IgG heavy chain Fc region extends from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore an antibody produced by a host cell by expression of a specific nucleic acid molecule encoding a full-length heavy chain may include the full-length heavy chain, or it may include a cleaved variant of the full-length heavy chain. This may be the case where the final two C-terminal amino acids of the heavy chain are glycine (G446) and lysine (K447, EU numbering system). Therefore, the C-terminal lysine (Lys447), or the C-terminal glycine (Gly446) and lysine (Lys447), of the Fc region may or may not be present. Amino acid sequences of heavy chains including an Fc region are denoted herein without C-terminal glycine-lysine dipeptide if not indicated otherwise. In one aspect, a heavy chain including an Fc region as specified herein, comprised in an antibody according to the invention, comprises an additional C-terminal glycine-lysine dipeptide (G446 and K447, EU numbering system). In one aspect, a heavy chain including an Fc region as specified herein, comprised in an antibody according to the invention, comprises an additional C-terminal glycine residue (G446, numbering according to EU index). Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

A “modification promoting the association of the first and the second subunit of the Fc domain” is a manipulation of the peptide backbone or the post-translational modifications of an Fc domain subunit that reduces or prevents the association of a polypeptide comprising the Fc domain subunit with an identical polypeptide to form a homodimer. A modification promoting association as used herein particularly includes separate modifications made to each of the two Fc domain subunits desired to associate (i.e. the first and the second subunit of the Fc domain), wherein the modifications are complementary to each other so as to promote association of the two Fc domain subunits. For example, a modification promoting association may alter the structure or charge of one or both of the Fc domain subunits so as to make their association sterically or electrostatically favorable, respectively. Thus, (hetero)dimerization occurs between a polypeptide comprising the first Fc domain subunit and a polypeptide comprising the second Fc domain subunit, which might be non-identical in the sense that further components fused to each of the subunits (e.g. antigen binding moieties) are not the same. In some embodiments the modification promoting association comprises an amino acid mutation in the Fc domain, specifically an amino acid substitution. In a particular embodiment, the modification promoting association comprises a separate amino acid mutation, specifically an amino acid substitution, in each of the two subunits of the Fc domain.

“Framework” or “FR” refers to variable domain residues other than complementary determining regions (CDRs). The FR of a variable domain generally consists of four FR domains: FR1, FR2, FR3, and FR4. Accordingly, the CDR and FR sequences generally appear in the following sequence in VH (or VL): FR1-CDR-H1(CDR-L1)-FR2-CDR-H2(CDR-L2)-FR3-CDR-H3(CDR-L3)-FR4.

The terms “full length antibody”, “intact antibody”, and “whole antibody” are used herein interchangeably to refer to an antibody having a structure substantially similar to a native antibody structure or having heavy chains that contain an Fc region as defined herein.

The terms “host cell”, “host cell line”, and “host cell culture” are used interchangeably and refer to cells into which exogenous nucleic acid has been introduced, including the progeny of such cells. Host cells include “transformants” and “transformed cells”, which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

The term “recombinant human antibody”, as used herein, is intended to include all human antibodies that are prepared, expressed, created or isolated by recombinant means, such as antibodies isolated from a host cell such as a NSO or CHO cell or from an animal (e.g. a mouse) that is transgenic for human immunoglobulin genes or antibodies expressed using a recombinant expression vector transfected into a host cell. Such recombinant human antibodies have variable and constant regions in a rearranged form. The recombinant human antibodies according to the invention have been subjected to in vivo somatic hypermutation. Thus, the amino acid sequences of the VH and VL regions of the recombinant antibodies are sequences that, while derived from and related to human germ line VH and VL sequences, may not naturally exist within the human antibody germ line repertoire in vivo.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda MD (1991), vols. 1-3. In one aspect, for the VL, the subgroup is subgroup kappa I as in Kabat et al., supra. In one aspect, for the VH, the subgroup is subgroup III as in Kabat et al., supra.

A “humanized” antibody refers to a chimeric antibody comprising amino acid residues from non-human CDRs and amino acid residues from human FRs. In certain aspects, a humanized antibody will comprise substantially all of at least one, and typically two, variable domains, in which all or substantially all of the CDRs correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR” as used herein refers to each of the regions of an antibody variable domain which are hypervariable in sequence and which determine antigen binding specificity, for example “complementarity determining regions” (“CDRs”).

Generally, antibodies comprise six CDRs: three in the VH (CDR-H1, CDR-H2, CDR-H3), and three in the VL (CDR-L1, CDR-L2, CDR-L3). Exemplary CDRs herein include:
(a) hypervariable loops occurring at amino acid residues 26-32 (L1), 50-52 (L2), 91-96 (L3), is 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991)); and
(c) antigen contacts occurring at amino acid residues 27c-36 (L1), 46-55 (L2), 89-96 (L3), 30-35b (H1), 47-58 (H2), and 93-101 (H3) (MacCallum et al. J. Mo. Biol. 262: 732-745 (1996)).

Unless otherwise indicated, the CDRs are determined according to Kabat et al., supra. One of skill in the art will understand that the CDR designations can also be determined according to Chothia, supra, McCallum, supra, or any other scientifically accepted nomenclature system.

An “immunoconjugate” is an antibody conjugated to one or more heterologous molecule(s), including but not limited to a cytotoxic agent.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain aspects, the individual or subject is a human.

An “isolated” antibody is one which has been separated from a component of its natural environment. In some aspects, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) methods. For a review of methods for assessment of antibody purity, see, e.g., Flatman et al., J. Chromatogr. B 848:79-87 (2007).

The term “nucleic acid molecule” or “polynucleotide” includes any compound and/or substance that comprises a polymer of nucleotides. Each nucleotide is composed of a base, specifically a purine- or pyrimidine base (i.e. cytosine (C), guanine (G), adenine (A), thymine (T) or uracil (U)), a sugar (i.e. deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. Herein, the term nucleic acid molecule encompasses deoxyribonucleic acid (DNA) including e.g., complementary DNA (cDNA) and genomic DNA, ribonucleic acid (RNA), in particular messenger RNA (mRNA), synthetic forms of DNA or RNA, and mixed polymers comprising two or more of these molecules. The nucleic acid molecule may be linear or circular. In addition, the term nucleic acid molecule includes both, sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA and RNA molecules which are suitable as a vector for direct expression of an antibody of the invention in vitro and/or in vivo, e.g., in a host or patient. Such DNA (e.g., cDNA) or RNA (e.g., mRNA) vectors, can be unmodified or modified. For example, mRNA can be chemically modified to enhance the stability of the RNA vector and/or expression of the encoded molecule so that mRNA can be injected into a subject to generate the antibody in vivo (see e.g., Stadler ert al, Nature Medicine 2017, published online 12 Jun. 2017, doi:10.1038/nm.4356 or EP 2 101 823 B1).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety (e.g., a cytotoxic moiety) or radiolabel. The naked antibody may be present in a pharmaceutical composition.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable domain (VH), also called a variable heavy domain or a heavy chain variable region, followed by three constant heavy domains (CH1, CH2, and CH3). Similarly, from N- to C-terminus, each light chain has a variable domain (VL), also called a variable light domain or a light chain variable region, followed by a constant light (CL) domain.

A “blocking” antibody or an “antagonist” antibody is one that inhibits or reduces a biological activity of the antigen it binds. In some embodiments, blocking antibodies or antagonist antibodies substantially or completely inhibit the biological activity of the antigen. For example, the anti-EGFR antibodies of the invention block the signaling through EGFR.

An “agonist” or activating antibody is one that enhances or initiates signaling by the antigen to which it binds. In some embodiments, agonist antibodies cause or activate signaling without the presence of the natural ligand.

The term “package insert” is used to refer to instructions customarily included in commercial packages of therapeutic products, that contain information about the indications, usage, dosage, administration, combination therapy, contraindications and/or warnings concerning the use of such therapeutic products.

“No substantial cross-reactivity” means that a molecule (e.g., an antibody) does not recognize or specifically bind an antigen different from the actual target antigen of the molecule (e.g. an antigen closely related to the target antigen), particularly when compared to that target antigen. For example, an antibody may bind less than about 10% to less than about 5% to an antigen different from the actual target antigen, or may bind said antigen different from the actual target antigen at an amount consisting of less than about 10%, 9%, 8% 7%, 6%, 5%, 4%, 3%, 2%, 1%, 0.5%, 0.2%, or 0.1%, preferably less than about 2%, 1%, or 0.5%, and most preferably less than about 0.2% or 0.1% antigen different from the actual target antigen.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity for the purposes of the alignment. Alignment for purposes of determining percent amino acid sequence identity can be achieved in various ways that are within the skill in the art, for instance, using publicly available computer software such as BLAST, BLAST-2, Clustal W, Megalign (DNASTAR) software or the FASTA program package. Those skilled in the art can determine appropriate parameters for aligning sequences, including any algorithms needed to achieve maximal alignment over the full length of the sequences being compared. Alternatively, the percent identity values can be generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087 and is described in WO 2001/007611.

Unless otherwise indicated, for purposes herein, percent amino acid sequence identity values are generated using the ggsearch program of the FASTA package version 36.3.8c or later with a BLOSUM50 comparison matrix. The FASTA program package was authored by W. R. Pearson and D. J. Lipman (1988), “Improved Tools for Biological Sequence Analysis”, PNAS 85:2444-2448; W. R. Pearson (1996) “Effective protein sequence comparison” Meth. Enzymol. 266:227-258; and Pearson et. al. (1997) Genomics 46:24-36 and is publicly available from www.fasta.bioch.virginia.edu/fasta_www2/fasta_down.shtml or www.ebi.ac.uk/Tools/sss/fasta. Alternatively, a public server accessible at fasta.bioch.virginia.edu/fasta_www2/index.cgi can be used to compare the sequences, using the ggsearch (global protein:protein) program and default options (BLOSUM50; open: −10; ext: −2; Ktup=2) to ensure a global, rather than local, alignment is performed. Percent amino acid identity is given in the output alignment header.

The term “pharmaceutical composition” or “pharmaceutical formulation” refers to a preparation which is in such form as to permit the biological activity of an active ingredient contained therein to be effective, and which contains no additional components which are unacceptably toxic to a subject to which the pharmaceutical composition would be administered.

As used herein, a “pharmaceutically acceptable carrier” or “pharmaceutically acceptable excipient” refers to an ingredient in a pharmaceutical composition or formulation, other than an active ingredient, which is nontoxic to a subject. Pharmaceutically acceptable carrier, for example, includes any and all material compatible with pharmaceutical administration including solvents, dispersion media, coatings, antibacterial and antifungal agents, isotonic and absorption delaying agents, and other materials and compounds compatible with pharmaceutical administration. Except insofar as any conventional media or agent is incompatible with the active compound, use thereof in the compositions of the invention is contemplated. Supplementary active compounds can also be incorporated into the compositions. In some examples, a pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some aspects, antibodies of the invention are used to delay development of a disease or to slow the progression of a disease.

The term “cancer” as used herein refers to proliferative diseases, such as the cancer is colorectal cancer, sarcoma, head and neck cancer, squamous cell carcinoma, breast cancer, pancreatic cancer, gastric cancer, thyroid cancer, non-small-cell lung carcinoma, small-cell lung cancer and mesothelioma, including refractory versions of any of the above cancers, or a combination of one or more of the above cancers. In one embodiment, the cancer is colorectal cancer.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three complementary determining regions (CDRs). (See, e.g., Kindt et al. Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “vector”, as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”.

As used herein, the term “antigen binding molecule” refers in its broadest sense to a molecule that specifically binds an antigenic determinant. Examples of antigen binding molecules are immunoglobulins and derivatives, e.g. fragments, thereof.

The term “antigen-binding site of an antibody” when used herein refer to the amino acid residues of an antibody which are responsible for antigen-binding. The antigen-binding portion of an antibody comprises amino acid residues from the “complementary determining regions” or “CDRs”. “Framework” or “FR” regions are those variable domain regions other than the hypervariable region residues as herein defined. Therefore, the light and heavy chain variable domains of an antibody comprise from N- to C-terminus the domains FR1, CDR1, FR2, CDR2, FR3, CDR3, and FR4. Especially, CDR3 of the heavy chain is the region which contributes most to antigen binding and defines the antibody's properties. CDR and FR regions are determined according to the standard definition of Kabat et al., Sequences of Proteins of Immunological Interest, 5th ed., Public Health Service, National Institutes of Health, Bethesda, MD (1991) and/or those residues from a “hypervariable loop”.

Antibody specificity refers to selective recognition of the antibody for a particular epitope of an antigen. Natural antibodies, for example, are monospecific. “Bispecific antibodies” according to the invention are antibodies which have two different antigen-binding specificities. Antibodies of the present invention are specific for two different antigens, i.e. DR5 as first antigen and FAP as second antigen.

The term “monospecific” antibody as used herein denotes an antibody that has one or more binding sites each of which bind to the same epitope of the same antigen.

The term “bispecific” means that the antigen binding molecule is able to specifically bind to at least two distinct antigenic determinants. Typically, a bispecific antigen binding molecule comprises at least two antigen binding sites, each of which is specific for a different antigenic determinant. In certain embodiments the bispecific antigen binding molecule is capable of simultaneously binding two antigenic determinants, particularly two antigenic determinants expressed on two distinct cells.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g. Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g. US 2006/0025576A1).

The antibody or fragment herein also includes a “Dual Acting FAb” or “DAF” comprising at least one antigen binding site that binds to FAP or DR5 as well as another, different antigen (see, US 2008/0069820, for example).

The term “valent” as used within the current application denotes the presence of a specified number of binding sites in an antibody molecule. As such, the terms “bivalent”, “tetravalent”, and “hexavalent” denote the presence of two binding sites, four binding sites, and six binding sites, respectively, in an antibody molecule. The bispecific antibodies according to the invention are at least “bivalent” and may be “trivalent” or “multivalent” (e.g. “tetravalent” or “hexavalent”).

Antibodies of the present invention have two or more binding sites and are bispecific. That is, the antibodies may be bispecific even in cases where there are more than two binding sites (i.e. that the antibody is trivalent or multivalent). Bispecific antibodies of the invention include, for example, multivalent single chain antibodies, diabodies and triabodies, as well as antibodies having the constant domain structure of full length antibodies to which further antigen-binding sites (e.g., single chain Fv, a VH domain and/or a VL domain, Fab, or (Fab)2) are linked via one or more peptide-linkers. The antibodies can be full length from a single species, or be chimerized or humanized.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The term “amino acid” as used within this application denotes the group of naturally occurring carboxy α-amino acids comprising alanine (three letter code: ala, one letter code: A), arginine (arg, R), asparagine (asn, N), aspartic acid (asp, D), cysteine (cys, C), glutamine (gln, Q), glutamic acid (glu, E), glycine (gly, G), histidine (his, H), isoleucine (ile, I), leucine (leu, L), lysine (lys, K), methionine (met, M), phenylalanine (phe, F), proline (pro, P), serine (ser, S), threonine (thr, T), tryptophan (trp, W), tyrosine (tyr, Y), and valine (val, V).

As used herein, the expressions “cell”, “cell line”, and “cell culture” are used interchangeably and all such designations include progeny. Thus, the words “transfectants” and “transfected cells” include the primary subject cell and cultures derived there from without regard for the number of transfers. It is also understood that all progeny may not be precisely identical in DNA content, due to deliberate or inadvertent mutations. Variant progeny that have the same function or biological activity as screened for in the originally transformed cell are included.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described in the following.

As used herein, the term “binding” or “specifically binding” refers to the binding of the antibody to an epitope of the antigen in an in-vitro assay, preferably in a surface plasmon resonance assay (SPR, BIAcore, GE-Healthcare Uppsala, Sweden). The affinity of the binding is defined by the terms ka (rate constant for the association of the antibody from the antibody/antigen complex), kD (dissociation constant), and KD (kD/ka). Binding or specifically binding means a binding affinity (KD) of 10⁻⁸ mol/1 or less, preferably 10⁻⁹ M to 10⁻¹³ mol/1.

Binding of the antibody to the death receptor can be investigated by a BIAcore assay (GE-Healthcare Uppsala, Sweden). The affinity of the binding is defined by the terms ka (rate constant for the association of the antibody from the antibody/antigen complex), kD (dissociation constant), and KD (kD/ka)

“Reduced binding”, for example reduced binding to an Fc receptor, refers to a decrease in affinity for the respective interaction, as measured for example by SPR. For clarity the term includes also reduction of the affinity to zero (or below the detection limit of the analytic method), i.e. complete abolishment of the interaction. Conversely, “increased binding” refers to an increase in binding affinity for the respective interaction.

“T cell activation” as used herein refers to one or more cellular response of a T lymphocyte, particularly a cytotoxic T lymphocyte, selected from: proliferation, differentiation, cytokine secretion, cytotoxic effector molecule release, cytotoxic activity, and expression of activation markers. Suitable assays to measure T cell activation are known in the art described herein.

A “target cell antigen” as used herein refers to an antigenic determinant presented on the surface of a target cell, for example a cell in a tumor such as a cancer cell or a cell of the tumor stroma. In particular “target cell antigen” refers to Folate Receptor 1.
As used herein, the terms “first” and “second” with respect to antigen binding moieties etc., are used for convenience of distinguishing when there is more than one of each type of moiety.

The term “epitope” includes any polypeptide determinant capable of specific binding to an antibody. In certain embodiments, epitope determinant include chemically active surface groupings of molecules such as amino acids, sugar side chains, phosphoryl, or sulfonyl, and, in certain embodiments, may have specific three dimensional structural characteristics, and or specific charge characteristics. An epitope is a region of an antigen that is bound by an antibody.

As used herein, the term “antigenic determinant” is synonymous with “antigen” and “epitope,” and refers to a site (e.g. a contiguous stretch of amino acids or a conformational configuration made up of different regions of non-contiguous amino acids) on a polypeptide macromolecule to which an antigen binding moiety binds, forming an antigen binding moiety-antigen complex. Useful antigenic determinants can be found, for example, on the surfaces of tumor cells, on the surfaces of virus-infected cells, on the surfaces of other diseased cells, on the surface of immune cells, free in blood serum, and/or in the extracellular matrix (ECM). The proteins referred to as antigens herein, e.g., EGFR, can be any native form the proteins from any vertebrate source, including mammals such as primates (e.g. humans) and rodents (e.g. mice and rats), unless otherwise indicated. In a particular embodiment the antigen is a human protein. Where reference is made to a specific protein herein, the term encompasses the “full-length”, unprocessed protein as well as any form of the protein that results from processing in the cell. The term also encompasses naturally occurring variants of the protein, e.g. splice variants or allelic variants.

As used herein, the terms “engineer, engineered, engineering,” particularly with the prefix “glyco-,” as well as the term “glycosylation engineering” are considered to include any manipulation of the glycosylation pattern of a naturally occurring or recombinant polypeptide or fragment thereof. Glycosylation engineering includes metabolic engineering of the glycosylation machinery of a cell, including genetic manipulations of the oligosaccharide synthesis pathways to achieve altered glycosylation of glycoproteins expressed in cells. Furthermore, glycosylation engineering includes the effects of mutations and cell environment on glycosylation. In one embodiment, the glycosylation engineering is an alteration in glycosyltransferase activity. In a particular embodiment, the engineering results in altered glucosaminyltransferase activity and/or fucosyltransferase activity.

The combination therapies in accordance with some embodiments of the invention have a synergistic effect. A “synergistic effect” of two compounds is one in which the effect of the combination of the two agents is greater than the sum of their individual effects and is statistically different from the controls and the single drugs.

### Combination Therapies

Provided herein are combination therapies (or compositions) comprising a compound of formula (I):

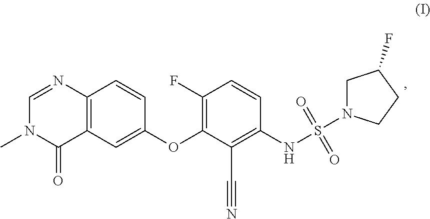 [SMILES: Cn1cnc2ccc(Oc3c(F)ccc(NS(=O)(=O)N4CC[C@@H](F)C4)c3C#N)cc2c1=O]

or a pharmaceutically acceptable salt thereof, and an EGFR inhibitor as described herein. In some embodiments, the compound of formula (I) is (3R)-N-[2-cyano-4-fluoro-3-(3-methyl-4-oxo-quinazolin-6-yl)oxy-phenyl]-3-fluoro-pyrrolidine-1-sulfonamide, or a pharmaceutically acceptable salt thereof. In some embodiments, the compound of formula (I) is a free base. In some embodiments, the compound of formula (I) is a pharmaceutically acceptable salt of the compound. In some embodiments, the compound of formula (I), or the pharmaceutically acceptable salt thereof, is a BRAF inhibitor.

Some embodiments of the invention include:

A combination of a BRAF inhibitor and an EGFR inhibitor wherein the BRAF inhibitor is a compound of formula (I)

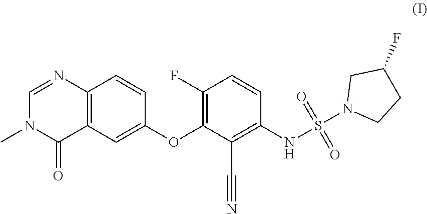 [SMILES: Cn1cnc2ccc(Oc3c(F)ccc(NS(=O)(=O)N4CC[C@@H](F)C4)c3C#N)cc2c1=O]

or a pharmaceutically acceptable salt thereof,

A combination of a BRAF inhibitor and an EGFR inhibitor wherein the BRAF inhibitor is a compound of formula (I)

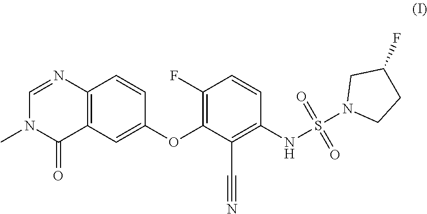 [SMILES: Cn1cnc2ccc(Oc3c(F)ccc(NS(=O)(=O)N4CC[C@@H](F)C4)c3C#N)cc2c1=O]

or a pharmaceutically acceptable salt thereof, wherein the EGFR inhibitor is cetuximab;
A combination of a BRAF inhibitor as described herein and an EGFR inhibitor, wherein the EGFR inhibitor is a monoclonal anti-EGFR antibody;
A combination of a BRAF inhibitor as described herein and an EGFR inhibitor, wherein the EGFR inhibitor is cetuximab or panitumumab;
A combination of a BRAF inhibitor as described herein and an EGFR inhibitor, wherein the EGFR inhibitor is cetuximab;
A combination of a BRAF inhibitor as described herein and an EGFR inhibitor, wherein the EGFR inhibitor is panitumumab;
A combination of a BRAF inhibitor and an EGFR inhibitor as described herein, for use as a medicament;
A combination of a BRAF inhibitor and an EGFR inhibitor as described herein, for use in the therapeutic and/or prophylactic treatment of cancer, in particular colorectal cancer;
A combination of a BRAF inhibitor and an EGFR inhibitor as described herein, for use in the therapeutic treatment of cancer, in particular colorectal cancer;
In a further embodiment the present invention relates to a pharmaceutical product for the treatment of colorectal cancer involving a tumor comprising b-Raf having the V600E mutation, comprising (A) a first component which comprises, as an active agent, Compound of formula (I), or a pharmaceutically-acceptable salt thereof; and (B) a second component which comprises, as an active agent, cetuximab; the amount of said active agents being such that the combination thereof is therapeutically-effective in the treatment of said cancer;
In one embodiment, cetuximab is administered initially as a 400 mg/m² dose as a 120-minute intravenous infusion, followed after a week by 250 mg/m² doses intravenously infused over 60 minutes once weekly. All agents may, for example, be administered until disease progression or unacceptable toxicity.
The use of a combination of a BRAF inhibitor and an EGFR inhibitor as described herein, for the preparation of a medicament for the treatment or prophylaxis of cancer, in particular colorectal cancer;
The use of a combination of a BRAF inhibitor and an EGFR inhibitor as described herein, for the preparation of a medicament for the treatment of cancer, in particular colorectal cancer;
A method for the treatment or prophylaxis of cancer, in particular colorectal cancer, the method comprising administering an effective amount of a combination of a BRAF inhibitor and an EGFR inhibitor as described herein, to a patient in need thereof, in particular wherein the compound of formula (I) or a pharmaceutically acceptable salt thereof, and cetuximab are administered at the same time, more particular wherein the compound of formula (I) and cetuximab are administered sequentially;
A method for the treatment of cancer, in particular colorectal cancer, the method comprising administering an effective amount of a combination of a BRAF inhibitor and an EGFR inhibitor as described herein, to a patient in need thereof, in particular wherein the compound of formula (I) or a pharmaceutically acceptable salt thereof, and cetuximab are administered at the same time, more particular wherein the compound of formula (I) and cetuximab are administered sequentially;
A method of treating a patient suffering from cancer, in particular colorectal cancer, the method comprising administering an effective amount of a combination of a BRAF inhibitor of formula (I) or a pharmaceutically acceptable salt thereor and an EGFR inhibitor as described herein, to a patient in need thereof, in particular wherein the compound of formula (I) and the EGFR inhibitor are administered at the same time, more particular wherein the compound of formula (I) and EGFR inhibitor are administered sequentially;
A method of treating a patient suffering from cancer, in particular colorectal cancer, the method comprising administering an effective amount of a combination of a BRAF inhibitor of formula (I) or a pharmaceutically acceptable salt thereof, and cetuximab, to a patient in need thereof, in particular wherein the compound of formula (I) and cetuximab are administered at the same time, more particular wherein the compound of formula (I) and cetuximab are administered sequentially;
A method of treating a patient suffering from cancer, in particular colorectal cancer, the method comprising administering an effective amount of a combination of a BRAF inhibitor of formula (I) or a pharmaceutically acceptable salt thereof, to a patient in need thereof, in particular wherein the compound of formula (I) and panitumumab are administered at the same time, more particular wherein the compound of formula (I) and panitumumab are administered sequentially;
A pharmaceutical composition comprising a combination of a BRAF inhibitor and an EGFR inhibitor as described herein and one or more pharmaceutically acceptable excipients;
A combination, a use, a method or a pharmaceutical composition as described herein, wherein the BRAF inhibitor is administered orally and the EGFR inhibitor is administered by injection, such as intravenous or subcutaneous injection;
A combination, a use, a method or a pharmaceutical composition as described herein, wherein the BRAF inhibitor is administered orally and the EGFR inhibitor is administered by intravenous injection;
A combination, a use, a method or a pharmaceutical composition as described herein, wherein the BRAF inhibitor is administered orally and the EGFR inhibitor is administered by subcutaneous injection;
A combination, a use, a method or a pharmaceutical composition according to any one of claims 1 to 9, wherein the BRAF inhibitor is administered concurrently with the EGFR inhibitor;
A combination, a use, a method or a pharmaceutical composition as described herein, wherein the BRAF inhibitor is administered sequentially with the EGFR inhibitor;
A combination of a BRAF inhibitor and an EGFR inhibitor for use as described herein, wherein the cancer is associated with BRAF^{V600X} mutations, in particular the BRAF^{V600E} or the BRAF^{V600K} mutation, more particular the BRAF^{V600E} mutation;
A combination of a BRAF inhibitor and an EGFR inhibitor for use as described herein, wherein the cancer is BRAF^{V600X} mutation-positive unresectable or metastatic cancer;
A combination of a BRAF inhibitor and an EGFR inhibitor for use as described herein, wherein the cancer is BRAF^{V600E} mutation-positive unresectable or metastatic cancer;
A combination of a BRAF inhibitor and an EGFR inhibitor for use as described herein, wherein the cancer is BRAF^{V600X} mutation-positive unresectable or metastatic colorectal cancer:
A combination of a BRAF inhibitor and an EGFR inhibitor for use as described herein, wherein the cancer is BRAF^{V600E} mutation-positive unresectable or metastatic colorectal cancer;
A combination BRAF inhibitor and an EGFR inhibitor for use as described herein, wherein BRAF^{V600X} mutation is determined using a method comprising (a) performing PCR or sequencing on nucleic acid (e.g., DNA) extracted from a sample of the patient's tumour tissue and/or body fluid; and (b) determining expression of BRAF^{V600} in the sample; and
A combination of a BRAF inhibitor and an EGFR inhibitor for use as described herein, comprising one or more additional anticancer agents selected from MEK inhibitors, MEK degraders, inhibitors of HER2 and/or HER3, degraders of HER2 and/or HER3, SHP2 inhibitors, SHP2 degraders, Axl inhibitors, Axl degraders, ALK inhibitors, ALK degraders, PI3K inhibitors, PI3K degraders, SOS1 inhibitors, SOS1 degraders, signal transduction patway inhibitors, checkpoint inhibitors, modulators of the apoptosis pathway, cytotoxic chemotherapeutics, angiogenesis-targeted therapies, immune-targeted agents, and antibody-drug conjugates.

Further embodiments of the invention include:

In one embodiment, a kit is provided, the kit comprising a BRAF inhibitor of formula (I) and an EGFR inhibitor as described therein, prescribing information also known as “leaflet”, a blister package or bottle (HDPE or glass) and a container. The prescribing information preferably includes the advice to a patient regarding the administration of the combination of the BRAF inhibitor and the EGFR inhibitor treatment as described herein;

In one embodiment, the treated subject became refractory to said prior treatment as described herein; and

In one embodiment, the treated subject developed brain metastasis during said prior treatment as described herein.

Structures depicted herein are also meant to include compounds that differ only in the presence of one or more isotopically enriched atoms. For example wherein one or more hydrogen atoms are replaced by deuterium (2H), or one or more carbon atoms are replaced by a ¹³C- or ¹⁴C-enriched carbon are within the scope of this invention.

Furthermore, embodiments of the invention include all optical isomers, i.e. diastereoisomers, diastereomeric mixtures, racemic mixtures, all their corresponding enantiomers and/or tautomers as well as their solvates, wherever applicable, of the compound of formula (I).

If desired, racemic mixtures of the compound of the invention may, for example, be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography.

In the embodiments, where an optically pure enantiomer is provided, optically pure enantiomer means that the compound contains >90% of the desired isomer by weight, particularly >95% of the desired isomer by weight, or more particularly >99% of the desired isomer by weight, said weight percent based upon the total weight of the isomer of the compound. A chirally pure or chirally enriched compound may be prepared by chirally selective synthesis or by separation of enantiomers. The separation of enantiomers may be carried out on the final product or alternatively on a suitable intermediate.

In one embodiment, one or more additional anticancer agents is used in combination with a BRAF inhibitor and an EGFR inhibitor as described herein, wherein the additional anticancer agents are selected from MEK inhibitors, MEK degraders, inhibitors of HER2 and/or HER3, degraders of HER2 and/or HER3, SHP2 inhibitors, SHP2 degraders, Axl inhibitors, Axl degraders, ALK inhibitors, ALK degraders, PI3K inhibitors, PI3K degraders, SOS1 inhibitors, SOS1 degraders, signal transduction patway inhibitors, checkpoint inhibitors, modulators of the apoptosis pathway, cytotoxic chemotherapeutics, angiogenesis-targeted therapies, immune-targeted agents, and antibody-drug conjugates.

In some embodiments, one of the additional anticancer agents is a MEK inhibitor. Non-limiting examples of MEK inhibitors include cobimetinib (Cotellic®), binimetinib (Mektovi®) and trametinib (Mekinist®). Additional examples of MEK inhibitors are known in the art.

In some embodiments, one of the additional anticancer agents is an inhibitor of HER2 and/or HER3. Non-limiting examples of HER2 and/or HER3 inhibitors include Iapatinib, canertinib, (E)-2-methoxy-N-(3-(4-(3-methyl-4-(6-methylpyridin-3-yloxy)phenylamino)quinazolin-6-yl)allyl)acetamide (GP-724714), sapitinib, 7-[[4-[(3-ethynylphenyl)amino]-7-methoxy-6-quinazolinyl]oxy]-N-hydroxy-heptanamide (CUDC-101), mubritinib, 6-[4-[(4-ethylpiperazin-1-yl)methyl]phenyl]-N-[(1R)-1-phenylethyl]-7H-pyrrolo[2,3-d]pyrimidin-4-amine (AEE788), irbinitinib (tucatinib), poziotinib, N-[4-[1-[4-(4-acetyl-1-piperazinyl)cyclohexyl]-4-amino-3-pyrazolo[3,4-d]pyrimidinyl]-2-methoxyphenyl]-1-methyl-2-indolecarboxamide (KIN001-111), 7-cyclopentyl-5-(4-phenoxyphenyl)-7H-pyrrolo[2,3-d]pyrimidin-4-ylamine (KIN001-051), 6,7-dimethoxy-N-(4-phenoxyphenyl)quinazolin-4-amine (KIN001-30), dasatinib, andbosutinib.

In some embodiments, one of the additional anticancer agents is an inhibitor of SHP2. Non-limiting examples of SHP2 inhibitors include 6-(4-amino-4-methylpiperidin-1-yl)-3-(2,3-dichlorophenyl)pyrazine-2-amine (SHP099), [3-[(3S,4S)-4-amino-3-methyl-2-oxa-8-azaspiro[4.5]decan-8-yl]-6-(2,3-dichlorophenyl)-5-methylpyrazin-2-yl]methanol (RMC-4550) RMC-4630, TNO155, and the compounds disclosed in WO 2015/107493, WO 2015/107494, WO 2015/107495, WO 2019/075265, PCT/U82019/056786 and PCT/182020/053019.

In some embodiments, one of the additional anticancer agents is a PI3K inhibitor. Non-limiting examples include buparlisib (BKM120), alpelisib (BYL719), samotolisib (LY3023414), 8-[(1R)-1-[(3,5-difluorophenyl)amino]ethyl]-N,N-dimethyl-2-(morpholin-4-yl)-4-oxo-4H-chromene-6-carboxamide (AZD8186), tenalisib (RP6530), voxtalisib hydrochloride (SAR-245409), gedatolisib (PF-05212384), panulisib (P-7170), taselisib (GDC-0032), trans-2-amino-8-[4-(2-hydroxyethoxy)cyclohexyl]-6-(6-methoxypyridin-3-yl)-4-methylpyrido[2,3-d]pyrimidin-7(8H)-one (PF-04691502), duvelisib (ABBV-954), N2-[4-oxo-4-[4-(4-oxo-8-phenyl-4H-1-benzopyran-2-yl)morpholin-4-ium-4-ylmethoxy]butyryl]-L-arginyl-glycyl-L-aspartyl-L-serine acetate (SF-1126), pictilisib (GDC-0941), 2-methyl-1-[2-methyl-3-(trifluoromethyl)benzyl]-6-(morpholin-4-yl)-1H-benzimidazole-4-carboxylic acid (GSK2636771), idelalisib (GS-1101), umbralisib tosylate (TGR-1202), pictilisib (GDC-0941), copanlisib hydrochloride (BAY 84-1236), dactolisib (BEZ-235), 1-(4-[5-[5-amino-6-(5-tert-butyl-1,3,4-oxadiazol-2-yl)pyrazin-2-yl]-1-ethyl-1-H-1,2,4-triazol-3-yl]piperidin-1-yl)-3-hydroxypropan-1-one (AZD-8835), 5-[6,6-dimethyl-4-(morpholin-4-yl)-8,9-dihydro-6H-[1,4]oxazino[4,3-e]purin-2-yl]pyrimidin-2-amine (GDC-0084) everolimus, rapamycin, perifosine, sirolimus and temsirolimus.

In some embodiments, one of the additional anticancer agents is an ALK inhibitor. Non-limiting examples include crizotinib (PF-02341066), ceritinib (LDK378), alectinib (alecensa), brigatinib (AP26113), lorlatinib (PF-6463922), ensartinib (X-396), entrectinib (RXDX-101), reprotectinib (TPX-0005), belizatinib (TSR-011), alkotinib (ZG-0418), foritinib (SAF-189), CEP-37440, TQ-B3139, PLB1003 and TPX-0131

In some embodiments, one of the additional anticancer agents is a checkpoint inhibitor. In some embodiments, the checkpoint inhibitor is a CTLA-4 inhibitor, a PD-1 binding antagonist or a PD-L1 binding antagonist. In some embodiments, the CTLA-4 inhibitor is ipilimumab (Yervoy®) or tremelimumab (GP-675,206). In some embodiments, the PD-1 binding antagonist is selected from cemiplimab (Libtayo®), pembrolizumab (Keytruda®), nivolumab (Opdivo®) and RN888 (PF-06801591). In some embodiments, the PD-L1 binding antagonist is selected from atezolizumab (Tecentriq®), avelumab (Bavencio®) and durvalumab (Imfinzi™)

In some embodiments, one of the additional anticancer agents is an antibody-drug conjugate. Non-limiting examples of an antibody-drug conjugate include gemtuzumab ozogamicin (Mylotarg™), inotuzumab ozogamicin (Besponsa®), brentuximab vedotin (Adcetris®), ado-trastuzumab emtansine (TDM-f, Kadcyla®), mirvetuximab soravtansine (IMGN853) and anetumab ravtansine.

In some embodiments, one of the additional anticancer agents is an antibody such as bevacizumab (Mvasti™, Avastin®), trastuzumab (Herceptin®), avelumab (Bavencio®), rituximab (MabThera™, Rituxan®), edrecolomab (Panorex), daratumuab (Darzalex®), olaratumab (Lartruvo™), ofatumumab (Arzerra®), alemtuzumab (Campath®), cetuximab (Erbitux®), oregovomab, cemiplimab (Libtayo®), pembrolizumab (Keytruda®), dinutiximab (Unituxin®), obinutuzumab (Gazyva®), tremelimumab (GP-675,206), ramucirumab (Cyramza®), ublituximab (TG-1101), panitumumab (Vectibix®), elotuzumab (EmplicitiT‘V’), necitumumab (PortrazzaT‘V’), cirmtuzumab (UC-961), ibritumomab (Zevalin®), isatuximab (SAR650984), nimotuzumab, fresolimumab (GC1008), Iirilumab (INN), mogamulizumab (Poteligeo®), ficlatuzumab (AV-299), denosumab (Xgeva®), ganitumab, urelumab, pidilizumab, amatuximab, blinatumomab (AMG103; Blincyto®) or midostaurin (Rydapt).

In another aspect provided herein is a method of treatment of colorectal cancer (CRC), in a patient in need thereof, the method comprising administering during a treatment period an effective amount of a combination therapy comprising (a) compound of formula (I), or a pharmaceutically acceptable salt thereof, and (b) an EGFR inhibitor, as described herein. In some embodiments, the colorectal cancer is EGFR-expressing, metastatic CRC or BRAF V600E Mutation-Positive Metastatic CRC. In some embodiments, the cancer is a cancer that acquired on-target EGFR resistance mutations. In a preferred aspect, the EGFR inhibitor is cetuximab or panitumumab. In a more preferred aspect, the EGFR inhibitor is cetuximab. In another more preferred aspect, the EGFR inhibitor is panitumumab.

### Pharmaceutical Formulations

Pharmaceutical formulations of antibodies, e.g., EGFR inhibitory antibodies as described herein are prepared by mixing such antibody having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

The formulation herein may also contain more than one active ingredients as necessary for the particular indication being treated, preferably those with complementary activities that do not adversely affect each other. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g. films, or microcapsules.

The formulations to be used for in vivo administration are generally sterile. Sterility may be readily accomplished, e.g., by filtration through sterile filtration membranes.

An antibody can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Another embodiment of the invention provides a pharmaceutical composition containing one or more compositions, wherein each composition contains one or more compounds for use according to the invention and one or more therapeutically inert carriers, diluents or excipients, as well as a method to prepare such a pharmaceutical compositions. In one example, the compound of formula (I) may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of formula (I) is formulated in an acetate buffer, at pH 5. In another embodiment, the compound of formula (I) is sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

Pharmaceutical compositions can be obtained by processing the BRAF inhibitor as described herein with pharmaceutically acceptable, inorganic or organic carriers or excipients. Lactose, corn starch or derivatives thereof, talc, stearic acids or it's salts and the like can be used, for example, as such carriers for tablets, coated tablets, dragées and hard gelatine capsules. Suitable carriers for soft gelatine capsules are, for example, vegetable oils, waxes, fats, semi-solid and liquid polyols and the like. Depending on the nature of the active substance no carriers are, however, usually required in the case of soft gelatine capsules. Suitable carriers for the production of solutions and syrups are, for example, water, polyols, glycerol, vegetable oil and the like. Suitable carriers for suppositories are, for example, natural or hardened oils, waxes, fats, semi-liquid or liquid polyols and the like.

The pharmaceutical compositions can, moreover, contain preservatives, solubilizers, stabilizers, wetting agents, emulsifiers, sweeteners, colorants, flavorants, salts for varying the osmotic pressure, buffers, masking agents or antioxidants. They can also contain still other therapeutically valuable substances.

Pharmaceutical compositions of a BRAF inhibitor, alone or in combination, can be prepared for storage by mixing the active ingredient having the desired degree of purity with optional pharmaceutically acceptable carriers, excipients or stabilizers (Remington's Pharmaceutical Sciences 16th edition, Osol, A. (ed.) (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

Pharmaceutical compositions of a BRAF inhibitor include those suitable for oral, nasal, topical (including buccal and sublingual), rectal, vaginal and/or parenteral administration. The compositions may conveniently be presented in unit dosage form and may be prepared by any methods well known in the art of pharmacy. The amount of active ingredient which can be combined with a carrier material to produce a single dosage form will vary depending upon the host being treated, as well as the particular mode of administration. The amount of active ingredient which can be combined with a carrier material to produce a single dosage form will generally be that amount of a BRAF inhibitor or an EGFR inhibitor treatment which produces a therapeutic effect. Generally, out of one hundred percent, this amount will range from about 1 percent to about 90 percent of active ingredient, preferably from about 5 percent to about 70 percent, most preferably from about 10 percent to about 30 percent. Methods of preparing these compositions include the step of bringing into association a BRAF inhibitor with the carrier and, optionally, one or more accessory ingredients. In general, the pharmaceutical compositions can be prepared by uniformaly and intimately bringing into association a BRAF inbitor with liquid carriers, or finely divided solid carriers, or both, and then, if necessary, shaping the product. Pharmaceutical compositions suitable for oral administration may be in the form of capsules, cachets, sachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a BRAF inhibitor as an active ingredient. A BRAF inhibitor may also be administered as a bolus, electuary or paste.

In one embodiment of the invention, a BRAF inhibitor and an EGFR inhibitor are formulated into two separate pharmaceutical compositions.

The active ingredients may also be entrapped in microcapsules prepared, for example, by coacervation techniques or by interracial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences, 16th edition, Osol, A. (ed.) (1980).

The formulations to be used for in vivo administration must be sterile. This can be readily accomplished by filtration through sterile filtration membranes.

The dosage can vary within wide limits and will, of course, have to be adjusted to the individual requirements in each particular case. In the case of oral administration the dosage for adults can vary from about 5 mg to about 4000 mg per day of a compound of general formula (I) or of the corresponding amount of a pharmaceutically acceptable solvate thereof. The daily dosage may be administered as single dose or in divided doses and, in addition, the upper limit can also be exceeded when this is found to be indicated.

The following examples illustrate the present invention without limiting it, but serve merely as representative thereof. The pharmaceutical compositions, such as for instance a tablet or a coated tablet, conveniently contain about 5 to 500 mg, particularly about 100 to 500 mg, of a compound of formula (I).

In some of the embodiments the compound of formula (I) is administered two times a day at a dose from about 500 mg to about 1000 mg, in particular at a dose of about 800 mg.

In some of the embodiments the compound of formula (I) is administered two times a day at a dose from about 1500 mg to about 2000 mg, in particular at a dose of about 1600 mg.

In some of the embodiments the compound of formula (I) is administered three times a day at a dose from about 1000 mg to about 1300 mg, in particular at a dose of about 1200 mg.

In one embodiment the compound of formula (I) is orally administered three times a day at a dose of about 1200 mg, while cetuximab is administered intravenously or subcutaneously at a dose of about 400 mg/m² to about 500 mg/m² as initial dose and then about 200 mg/m² to about 300 mg/m² once per week.

In one embodiment the compound of formula (I) is orally administered three times a day at a dose of about 1200 mg, while cetuximab is administered intravenously or subcutaneously at a dose of about 400 mg/m² as initial dose and then about 250 mg/m² once per week.

In one embodiment the compound of formula (I) is orally administered three times a day at a dose of about 1200 mg, while cetuximab is administered intravenously or subcutaneously at a dose of about 400 mg/m² as initial dose and then about 250 mg/m² once per week, and while FOLFOX or FOLFIRI chemotherapy is also administered every second week.

In one embodiment the compound of formula (I) is orally administered three times a day at a dose of about 1200 mg, while cetuximab is administered intravenously or subcutaneously at a dose of about 400 mg/m² as initial dose and then about 500 mg/m² once every other week, while FOLFOX or FOLFIRI chemotherapy is also administered every second week.

In one embodiment the compound of formula (I) is orally administered three times a day at a dose of about 1200 mg, while cetuximab is administered intravenously or subcutaneously at a dose of about 400 mg/m² as initial dose and then about 250 mg/m² once per week, and while FOLFOX chemotherapy is also administered every second week.

In one embodiment the compound of formula (I) is orally administered three times a day at a dose of about 1200 mg, while cetuximab is administered intravenously or subcutaneously at a dose of about 400 mg/m² as initial dose and then about 250 mg/m² once per week, and while FOLFIRI chemotherapy is also administered every second week.

In one embodiment the compound of formula (I) is orally administered two times a day at a dose of about 800 mg, while cetuximab is administered intravenously or subcutaneously at a dose of about 400 mg/m² as initial dose and then about 250 mg/m² once per week.

In one embodiment the compound of formula (I) is orally administered two times a day at a dose of about 800 mg, while cetuximab is administered intravenously or subcutaneously at a dose of about 400 mg/m² to about 500 mg/m² as initial dose and then about 200 mg/m² to about 300 mg/m² once per week.

In one embodiment the compound of formula (I) is orally administered two times a day at a dose of about 800 mg, while cetuximab is administered intravenously or subcutaneously at a dose of about 400 mg/m² as initial dose and then about 250 mg/m² once per week.

In one embodiment the compound of formula (I) is orally administered two times a day at a dose of about 1600 mg, while cetuximab is administered intravenously or subcutaneously at a dose of about 400 mg/m² to about 500 mg/m² as initial dose and then about 200 mg/m² to about 300 mg/m² once per week.

In one embodiment the compound of formula (I) is orally administered two times a day at a dose of about 1600 mg, while cetuximab is administered intravenously or subcutaneously at a dose of about 400 mg/m² as initial dose and then about 250 mg/m² once per week every second week.

In one embodiment the compound of formula (I) is orally administered two times a day at a dose of about 1600 mg, while cetuximab is administered intravenously or subcutaneously at a dose of about 400 mg/m² as initial dose and then about 250 mg/m² once per week, and while FOLFOX or FOLFIRI chemotherapy is also administered every second week.

In one embodiment the compound of formula (I) is orally administered two times a day at a dose of about 1600 mg, while cetuximab is administered intravenously or subcutaneously at a dose of about 400 mg/m² as initial dose and then about 500 mg/m² once every other week, while FOLFOX or FOLFIRI chemotherapy is also administered every second week.

In one embodiment the compound of formula (I) is orally administered two times a day at a dose of about 1600 mg, while cetuximab is administered intravenously or subcutaneously at a dose of about 400 mg/m² as initial dose and then about 250 mg/m² once per week, and while FOLFOX chemotherapy is also administered every second week.

In one embodiment the compound of formula (I) is orally administered two times a day at a dose of about 1600 mg, while cetuximab is administered intravenously or subcutaneously at a dose of about 400 mg/m² as initial dose and then about 250 mg/m² once per week, and while FOLFIRI chemotherapy is also administered every second week.

In one embodiment the compound of formula (I) is orally administered two times a day at a dose of about 1800 mg, while cetuximab is administered intravenously or subcutaneously at a dose of about 400 mg/m² as initial dose and then about 250 mg/m² once per week.

In one embodiment the compound of formula (I) is orally administered two times a day at a dose of about 1800 mg, while cetuximab is administered intravenously or subcutaneously at a dose of about 400 mg/m² as initial dose and then about 250 mg/m² once per week, and while FOLFOX or FOLFIRI chemotherapy is also administered every second week.

In one embodiment the compound of formula (I) is orally administered two times a day at a dose of about 1800 mg, while cetuximab is administered intravenously or subcutaneously at a dose of about 400 mg/m² as initial dose and then about 500 mg/m² once every other week, while FOLFOX or FOLFIRI chemotherapy is also administered every second week.

In one embodiment the compound of formula (I) is orally administered two times a day at a dose of about 1800 mg, while cetuximab is administered intravenously or subcutaneously at a dose of about 400 mg/m² as initial dose and then about 250 mg/m² once per week, and while FOLFOX chemotherapy is also administered every second week.

In one embodiment the compound of formula (I) is orally administered two times a day at a dose of about 1800 mg, while cetuximab is administered intravenously or subcutaneously at a dose of about 400 mg/m² as initial dose and then about 250 mg/m² once per week, and while FOLFIRI chemotherapy is also administered every second week.

In one embodiment the compound of formula (I) is orally administered two times a day at a dose of about 2000 mg, while cetuximab is administered intravenously or subcutaneously at a dose of about 400 mg/m² as initial dose and then about 250 mg/m² once per week.

In one embodiment the compound of formula (I) is orally administered two times a day at a dose of about 2000 mg, while cetuximab is administered intravenously or subcutaneously at a dose of about 400 mg/m² as initial dose and then about 250 mg/m² once per week, and while FOLFOX chemotherapy is also administered every second week.

In one embodiment the compound of formula (I) is orally administered two times a day at a dose of about 2000 mg, while cetuximab is administered intravenously or subcutaneously at a dose of about 400 mg/m² as initial dose and then about 250 mg/m² once per week, and while FOLFOX or FOLFIRI chemotherapy is also administered every second week.

In one embodiment the compound of formula (I) is orally administered two times a day at a dose of about 2000 mg, while cetuximab is administered intravenously or subcutaneously at a dose of about 400 mg/m² as initial dose and then about 500 mg/m² once every other week, while FOLFOX or FOLFIRI chemotherapy is also administered every second week.

In one embodiment the compound of formula (I) is orally administered two times a day at a dose of about 2000 mg, while cetuximab is administered intravenously or subcutaneously at a dose of about 400 mg/m² as initial dose and then about 250 mg/m² once per week, and while FOLFIRI chemotherapy is also administered every second week.

In one embodiment the compound of formula (I) is orally administered three times a day at a dose of about 1200 mg, while panitumumab is administered intravenously at a dose of about 6 mg/kg as initial dose and then about 6 mg/kg once every two weeks (q14 days).

In one embodiment the compound of formula (I) is orally administered three times a day at a dose of about 1200 mg, while panitumumab is administered intravenously at a dose of about 6 mg/kg as initial dose and then about 6 mg/kg once every two weeks (q14 days), while FOLFOX or FOLFIRI chemotherapy is also administered every two weeks.

In one embodiment the compound of formula (I) is orally administered two times a day at a dose of about 1600 mg, while panitumumab is administered intravenously at a dose of about 6 mg/kg as initial dose and then about 6 mg/kg once every two weeks (q14 days).

In one embodiment the compound of formula (I) is orally administered two times a day at a dose of about 1600 mg, while panitumumab is administered intravenously at a dose of about 6 mg/kg as initial dose and then about 6 mg/kg once every two weeks (q14 days), while FOLFOX or FOLFIRI chemotherapy is also administered every two weeks.

In one embodiment the compound of formula (I) is orally administered two times a day at a dose of about 1800 mg, while panitumumab is administered intravenously at a dose of about 6 mg/kg as initial dose and then about 6 mg/kg once every two weeks (q14 days).

In one embodiment the compound of formula (I) is orally administered two times a day at a dose of about 1800 mg, while panitumumab is administered intravenously at a dose of about 6 mg/kg as initial dose and then about 6 mg/kg once every two weeks (q14 days), while FOLFOX or FOLFIRI chemotherapy is also administered every two weeks.

In one embodiment the compound of formula (I) is orally administered two times a day at a dose of about 2000 mg, while panitumumab is administered intravenously at a dose of about 6 mg/kg as initial dose and then about 6 mg/kg once every two weeks (q14 days).

In one embodiment the compound of formula (I) is orally administered two times a day at a dose of about 2000 mg, while panitumumab is administered intravenously at a dose of about 6 mg/kg as initial dose and then about 6 mg/kg once every two weeks (q14 days), while FOLFOX or FOLFIRI chemotherapy is also administered every two weeks.

### EXAMPLES

The following examples are provided to illustrate the invention and have no limiting character. Examples of compositions according to the invention include:

### Example A

Tablets of the following composition are manufactured in the usual manner.

TABLE 3
Possible Tablet Composition

| | mg/tablet | | | |
|---|---|---|---|---|
| Ingredient | 5 | 25 | 100 | 500 |
| Compound of formula (I) | 5 | 25 | 100 | 500 |
| Lactose Anhydrous DTG | 125 | 105 | 30 | 150 |
| Sta-Rx 1500 | 6 | 6 | 6 | 60 |
| Microcrystalline Cellulose | 30 | 30 | 30 | 450 |
| Magnesium Stearate | 1 | 1 | 1 | 1 |
| Total | 167 | 167 | 167 | 831 |

### Manufacturing Procedure

1 Mix ingredients 1, 2, 3 and 4 and granulate with purified water.
2. Dry the granules at 50° C.
3. Pass the granules through suitable milling equipment.
4. Add ingredient 5 and mix for three minutes; compress on a suitable press.

### Example B-1

Capsules of the following composition are manufactured.

TABLE 4
Possible Capsule Ingredient Composition

| | mg/capsule | | | |
|---|---|---|---|---|
| Ingredient | 5 | 25 | 100 | 500 |
| Compound of formula (I) | 5 | 25 | 100 | 500 |
| Hydrous Lactose | 159 | 123 | 148 | — |
| Corn Starch | 25 | 35 | 40 | 70 |
| Talk | 10 | 15 | 10 | 25 |
| Magnesium Stearate | 1 | 2 | 2 | 5 |
| Total | 200 | 200 | 300 | 600 |

### Manufacturing Procedure

1. Mix ingredients 1, 2 and 3 in a suitable mixer for 30 minutes.
2. Add ingredients 4 and 5 and mix for 3 minutes.
3. Fill into a suitable capsule.

The compound of formula (I), lactose and corn starch are firstly mixed in a mixer and then in a comminuting machine. The mixture is returned to the mixer; the talc is added thereto and mixed thoroughly. The mixture is filled by machine into suitable capsules, e.g. hard gelatin capsules.

### Example B-2

Soft Gelatin Capsules of the following composition are manufactured.

TABLE 5
Possible Soft Gelatin Capsule Ingredient Composition

| Ingredient | mg/capsule |
|---|---|
| Compound of formula (I) | 5 |
| Yellow wax | 8 |
| Hydrogenated Soya bean oil | 8 |
| Partially hydrogenated plant oils | 34 |
| Soya bean oil | 110 |
| Total | 165 |

TABLE 6
Possible Soft Gelatin Capsule Composition

| Ingredient | mg/capsule |
|---|---|
| Gelatin | 75 |
| Glycerol 85% | 32 |
| Karion 83 | 8 (dry matter) |
| Titan dioxide | 0.4 |
| Iron oxide yellow | 1.1 |
| Total | 116.5 |

### Manufacturing Procedure

The compound of formula (I) is dissolved in a warm melting of the other ingredients and the mixture is filled into soft gelatin capsules of appropriate size. The filled soft gelatin capsules are treated according to the usual procedures.

### Example C

Suppositories of the following composition are manufactured:

TABLE 7
Possible Suppository Composition

| Ingredient | mg/supp. |
|---|---|
| Compound of formula (I) | 15 |
| Suppository mass | 1285 |
| Total | 1300 |

### Manufacturing Procedure

The suppository mass is melted in a glass or steel vessel, mixed thoroughly and cooled to 45° C. Thereupon, the finely powdered compound of formula (I) is added thereto and stirred until it has dispersed completely. The mixture is poured into suppository moulds of suitable size, left to cool; the suppositories are then removed from the moulds and packed individually in wax paper or metal foil.

### Example D

Injection solutions of the following composition are manufactured.

TABLE 8
Possible Injection Solution Composition

| Ingredient | mg/injection solution. |
|---|---|
| Compound of formula (I) | 3 |
| Polyethylene Glycol 400 | 150 |
| acetic acid | q.s. ad pH 5.0 |
| water for injection solutions | ad 1.0 ml |

### Manufacturing Procedure

The compound of formula (I) is dissolved in a mixture of Polyethylene Glycol 400 and water for injection (part). The pH is adjusted to 5.0 by acetic acid. The volume is adjusted to 1.0 ml by addition of the residual amount of water. The solution is filtered, filled into vials using an appropriate overage and sterilized.

### Example E

Sachets of the following composition are manufactured.

TABLE 9
Possible Sachet Composition

| Ingredient | mg/sachet |
|---|---|
| Compound of formula (I) | 50 |
| Lactose, fine powder | 1015 |
| Microcrystalline cellulose (AVICEL PH 102) | 1400 |
| Sodium carboxymethyl cellulose | 14 |
| Polyvinylpyrrolidon K 30 | 10 |
| Magnesium stearate | 10 |
| Flavoring additives | 1 |
| Total | 2500 |

### Manufacturing Procedure

The compound of formula (I) is mixed with lactose, microcrystalline cellulose and sodium carboxymethyl cellulose and granulated with a mixture of polyvinylpyrrolidone in water. The granulate is mixed with magnesium stearate and the flavoring additives and filled into sachets.

### Biological Examples

### Test Agents

(3R)-N-[2-cyano-4-fluoro-3-(3-methyl-4-oxo-quinazolin-6-yl)oxy-phenyl]-3-fluoro-pyrrolidine-1-sulfonamide (herein referred to as R07276389) was provided as a powder from Roche, Basel, Switzerland and resuspended prior to use. Cetuximab was provided by WuXi (China).

### Cell Line and Culture Conditions

Cell lines were obtained from ATCC, maintained in humidified incubators at 5% CO2 in standard conditions and passaged twice a week. Culture conditions are reported in the following table:

| Cell line | catalog#/origin | culture condition |
|---|---|---|
| LS411 | ATCC cat CRL- | RPMI-1640 Medium + |
| | 2159 | 10% Fetal Calf |
| | | Serum + 5% Pen/Strep |
| HT29 | ATCC cat | McCoy's 5a Medium + |
| | HTB-38 | 10% Fetal Calf |
| | | Serum + 5% Pen/Strep |

### Experimental Procedure

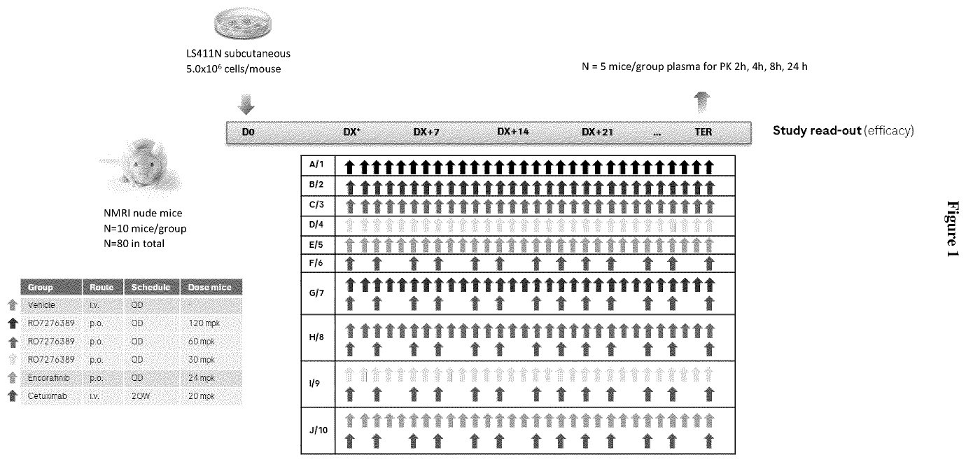

Experiments were conducted at WuXi (China) who provided female BALB/c Nude mice and Cexutimab. Mice were implanted subcutaneously on one flank and at tumor establishment (between 100 and 200 mm³) mice were randomized and treated daily orally with R07276389 or biweekly intravenously with Cetuximab as reported in FIG. 1

### Example 1

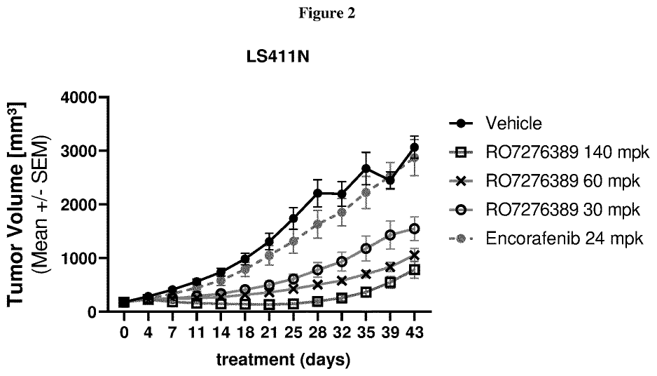

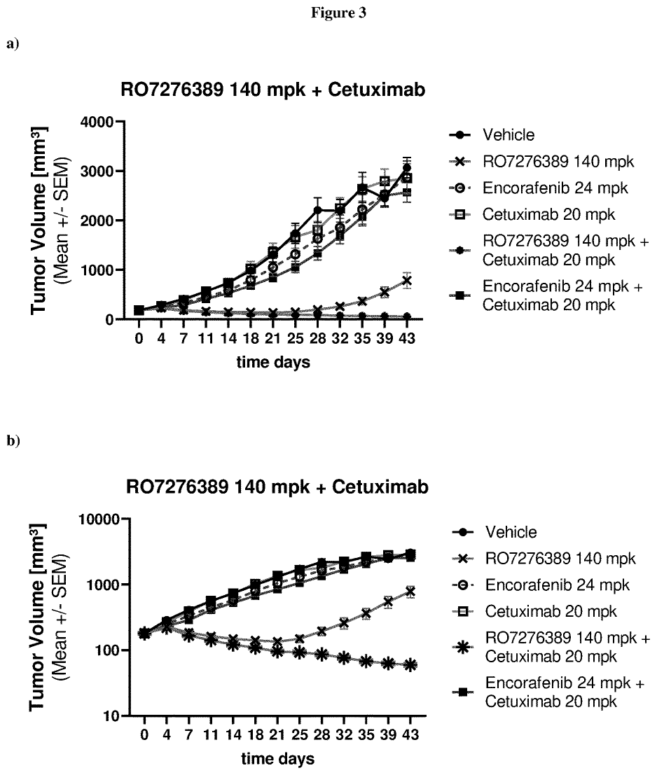

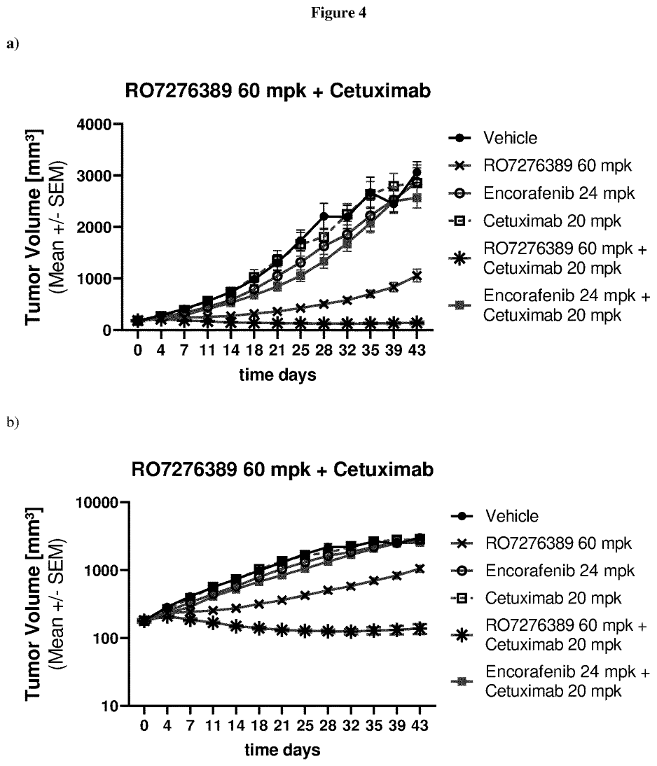

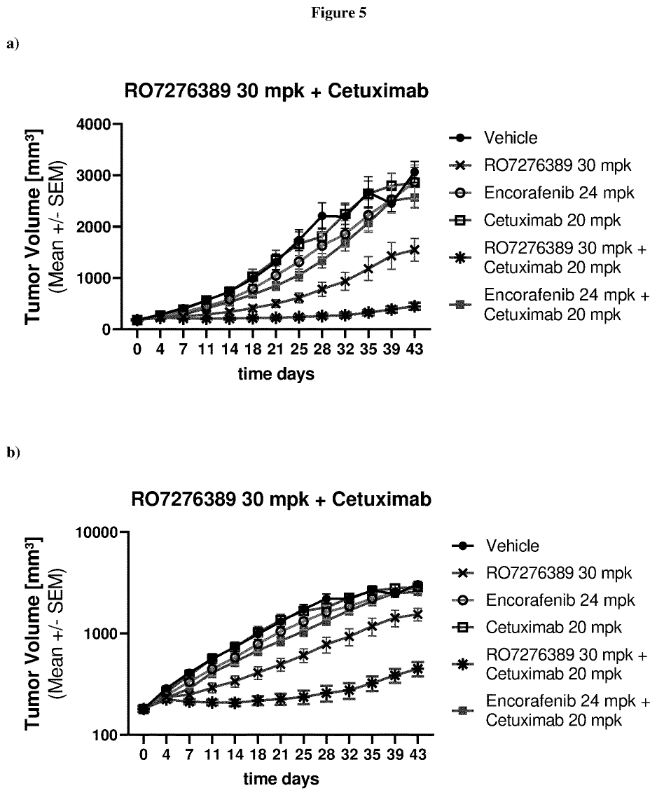

Mice were implanted with the cell line LS411N harboring the BRAF V600E mutation (5.0×10⁶ cells/mouse). Upon tumor establishment (150 mm³) mice were randomized and received orally (PO) once per day (QD: quaque die; once a day) either R07276389 (140 mpk, 60 mpk or mpk) or encorafenib (24 mpk), alone or in combination with cetuximab via IV injection (2QW). A schematic representation of the study design is shown in FIG. 1. Results reported in FIG. 2 evidenced that R07276389 monotherapy achieved a dose-dependent tumour growth inhibition that was significantly higher when compared with encorafenib. Results reported in FIGS. 3 to 5 evidenced that the combination of R07276389 and EGFRi has the potential to significantly reduce tumour growth in CRC when compared with a combination of the current standard of care (SOC) encorafenib in combination with cetuximab. Remarkably, treatment with the higher doses of 140 mpk and 60 mpk R07276389 in combination with cetuximab even resulted in a reduction of the tumour volume.

### Example 2

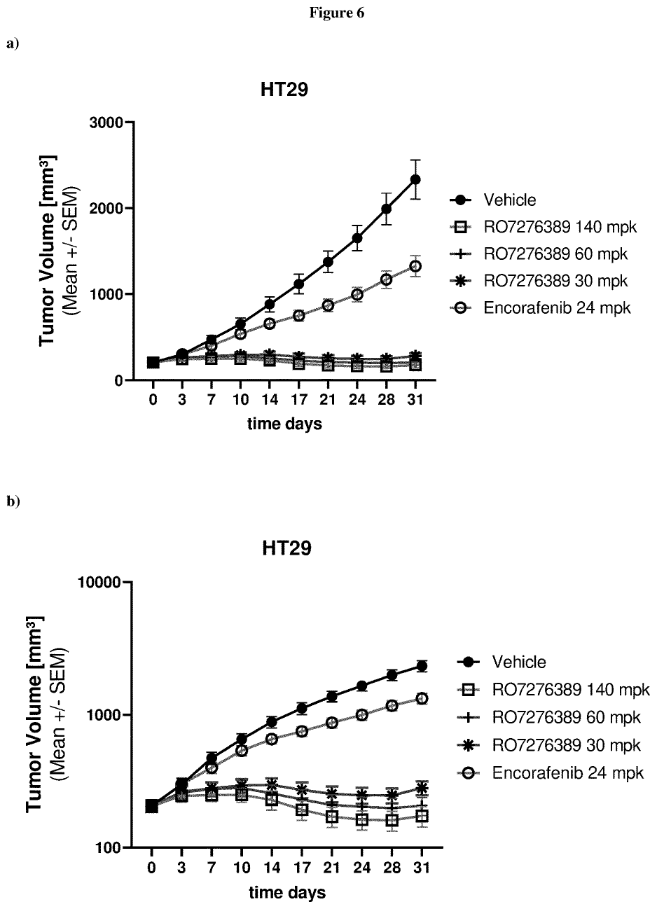

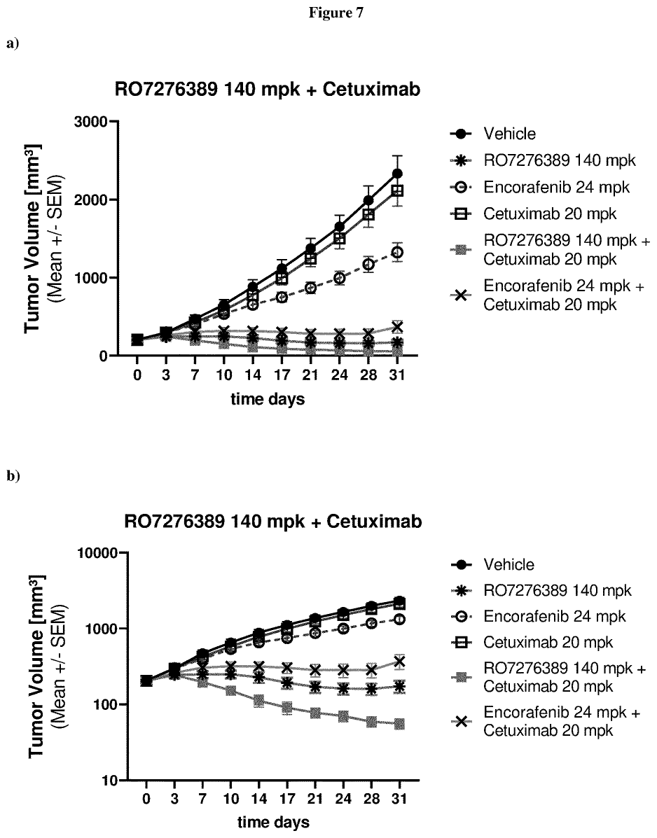

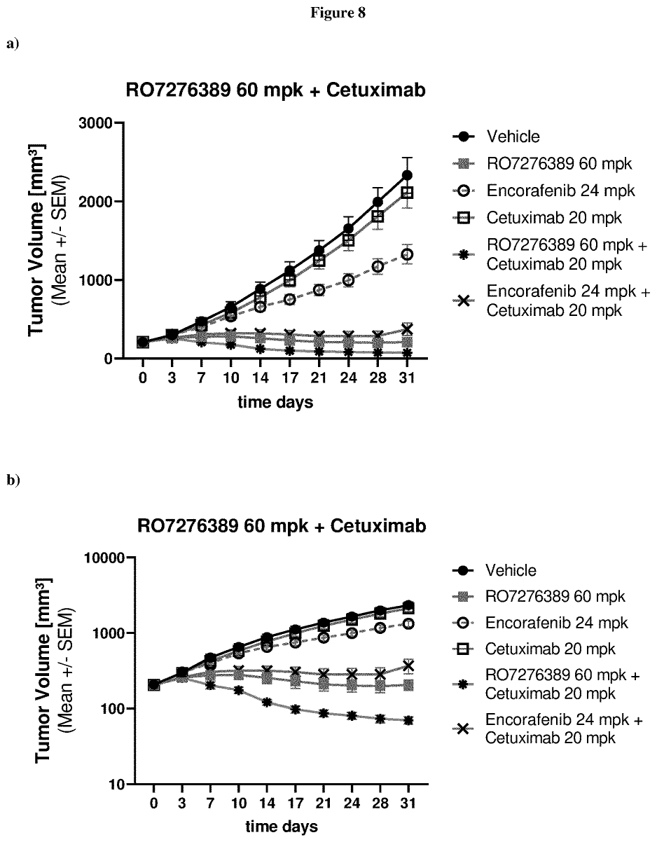

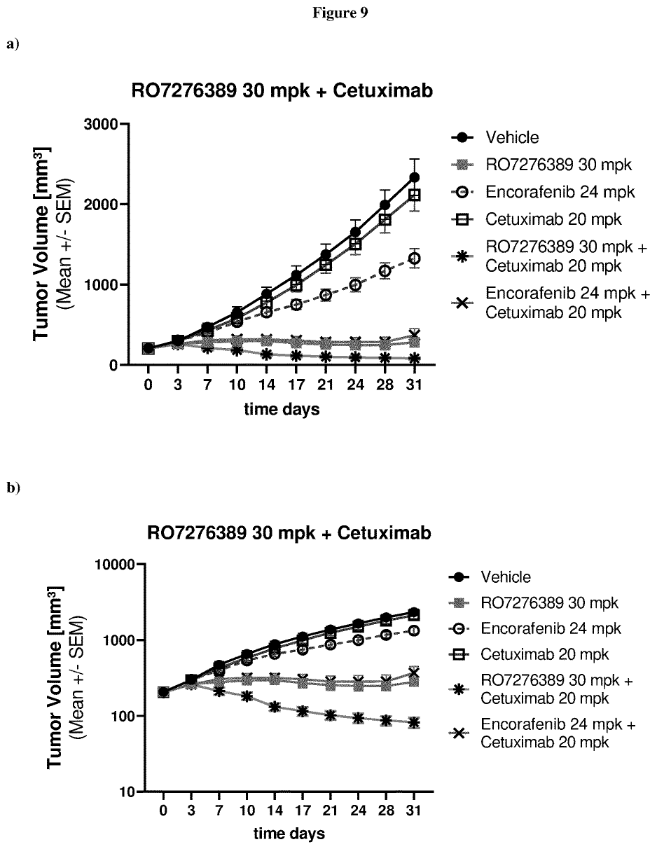

Mice were implanted with the cell line HT29 harboring the BRAF V600E mutation (5.0×10⁶ cells/mouse). Upon tumor establishment (approximately 150 mm³) mice were randomized and received orally (PO) once per day (QD) either R07276389 (140 mpk, 60 mpk or 30 mpk) or encorafenib (24 mpk), alone or in combination with cetuximab via IV injection (2QW). Results reported in FIG. 6 evidenced that R07276389 monotherapy achieved a dose-dependent tumour growth inhibition that was significantly higher when compared with encorafenib. Results reported in FIGS. 7 to 9 evidenced that the combination of R07276389 and EGFRi has the potential to significantly reduce tumour growth in CRC when compared with a combination of the current SOC encorafenib in combination with cetuximab. Remarkably, treatment with R07276389 in combination with cetuximab even resulted in a reduction of the tumour volume.

1-20. (canceled)

21. A method of treating cancer, the method comprising administering to a patient in need thereof an effective amount of a BRAF inhibitor of formula (I): 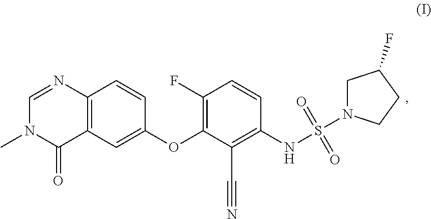 [SMILES: Cn1cnc2ccc(Oc3c(F)ccc(NS(=O)(=O)N4CC[C@@H](F)C4)c3C#N)cc2c1=O]
or a pharmaceutically acceptable salt thereof,
in combination with an effective amount of an EGFR inhibitor, or a pharmaceutically acceptable salt thereof.

22. The method of claim 21, wherein the compound of formula (I) is (3R)-N-[2-cyano-4-fluoro-3-(3-methyl-4-oxo-quinazolin-6-yl)oxy-phenyl]-3-fluoro-pyrrolidine-1-sulfonamide, or a pharmaceutically acceptable salt thereof.

23. The method of claim 21, wherein the compound of formula (I) is a free base.

24. The method of claim 21, wherein the EGFR inhibitor is a monoclonal anti-EGFR antibody.

25. The method of claim 24, wherein the EGFR inhibitor is cetuximab.

26. The method of claim 21, wherein the cancer is colorectal cancer.

27. The method of claim 21, wherein the cancer is associated with a BRAF^{V600X} mutation.

28. The method of claim 27, wherein BRAF^{V600X} mutation is determined using a method comprising (a) performing PCR or sequencing on DNA extracted from a sample of the patient's tumor tissue and/or bodily fluid; and (b) determining expression of BRAF^{V600} in the sample.

29. The method of claim 27, wherein the cancer is BRAF^{V600X} mutation-positive unresectable or metastatic cancer.

30. The method of claim 27, wherein the cancer is associated with the BRAF^{V600E} mutation.

31. The method of claim 30, wherein BRAF^{V600E} mutation-positive unresectable or metastatic cancer.

32. The method of claim 26, wherein the cancer is BRAF^{V600E} mutation-positive unresectable or metastatic colorectal cancer.

33. The method of claim 21, wherein the BRAF inhibitor of formula (I), or a pharmaceutically acceptable salt thereof, and the EGFR inhibitor, or a pharmaceutically acceptable salt thereof, are administered to the patient concurrently.

34. The method of claim 21, wherein the BRAF inhibitor of formula (I), or a pharmaceutically acceptable salt thereof, and the EGFR inhibitor, or a pharmaceutically acceptable salt thereof, are administered to the patient sequentially.

35. The method of claim 21, the BRAF inhibitor of formula (I), or a pharmaceutically acceptable salt thereof, is administered to the patient orally, and the EGFR inhibitor, or a pharmaceutically acceptable salt thereof, is administered to the patient by an intravenous or a subcutaneous injection.

36. The method of claim 21, further comprising administering to the patient one or more additional anticancer agents selected from MEK inhibitors, MEK degraders, inhibitors of HER2 and/or HER3, degraders of HER2 and/or HER3, SHP2 inhibitors, SHP2 degraders, Axl inhibitors, Axl degraders, ALK inhibitors, ALK degraders, PI3K inhibitors, PI3K degraders, SOS1 inhibitors, SOS1 degraders, signal transduction pathway inhibitors, checkpoint inhibitors, modulators of the apoptosis pathway, cytotoxic chemotherapeutics, angiogenesis-targeted therapies, immune-targeted agents, and antibody-drug conjugates.

37. The method of claim 36, comprising administering the patient an effective amount of FOLFOX chemotherapy or FOLFIRI chemotherapy.

38. A method of treating BRAF^{V600E} mutation-positive unresectable or metastatic colorectal cancer, the method comprising administering to a patient in need thereof an effective amount of a BRAF inhibitor of formula (I): 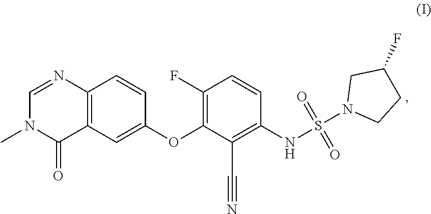 [SMILES: Cn1cnc2ccc(Oc3c(F)ccc(NS(=O)(=O)N4CC[C@@H](F)C4)c3C#N)cc2c1=O]
or a pharmaceutically acceptable salt thereof,
in combination with an effective amount of a monoclonal anti-EGFR antibody.

39. The method of claim 38, wherein:
the anti-EGFR antibody is cetuximab,
the BRAF inhibitor of formula (I), or a pharmaceutically acceptable salt thereof, is administered to the patient orally and the cetuximab is administered to the patient by intravenous or subcutaneous injection, and
the BRAF inhibitor of formula (I), or a pharmaceutically acceptable salt thereof, and cetuximab are administered to the patient concurrently.

40. The method of claim 38, wherein:
the anti-EGFR antibody is cetuximab,
the BRAF inhibitor of formula (I), or a pharmaceutically acceptable salt thereof, is administered to the patient orally and the cetuximab is administered to the patient by intravenous or subcutaneous injection, and
the BRAF inhibitor of formula (I), or a pharmaceutically acceptable salt thereof, and cetuximab are administered to the patient sequentially.
